(19) United States
(12) Patent Application Publication
(10) Pub. No.: US 2024/0372144 A1
(43) Pub. Date: Nov. 7, 2024

(54) ELECTROLYTIC SOLUTION FOR SECONDARY BATTERY, AND SECONDARY BATTERY

(71) Applicant: MURATA MANUFACTURING CO., LTD., Kyoto (JP)

(72) Inventor: Masayuki IHARA, Kyoto (JP)

(21) Appl. No.: 18/767,483

(22) Filed: Jul. 9, 2024

Related U.S. Application Data
(63) Continuation of application No. PCT/JP2023/005459, filed on Feb. 16, 2023.

(30) Foreign Application Priority Data
Feb. 25, 2022 (JP) 2022-028424

(51) Int. Cl. H01M 10/0567; C07C 317/08; C07D 327/00; C07D 327/04; C07D 327/10; H01M 10/0525; H01M 10/0568; H01M 10/42

(52) U.S. Cl. CPC H01M 10/0567; C07C 317/08; C07D 327/00; C07D 327/04; C07D 327/10; H01M 10/0525; H01M 10/0568; H01M 10/4235; H01M 2300/0025

(57) ABSTRACT

A secondary battery includes a positive electrode, a negative electrode, and an electrolytic solution. The electrolytic solution includes an electrolyte salt and a sulfinyl compound. The electrolyte salt includes an imide anion, and the imide anion includes at least one of an anion represented by Formula (1), an anion represented by Formula (2), an anion represented by Formula (3), or an anion represented by Formula (4). The sulfinyl compound includes at least one of a compound represented by Formula (5), a compound represented by Formula (6), a compound represented by Formula (7), a compound represented by Formula (8), a compound represented by Formula (9), a compound represented by Formula (10), or a compound represented by Formula (11).

### CROSS REFERENCE TO RELATED APPLICATIONS

The present application is a continuation of PCT patent application no. PCT/JP2023/005459, filed on Feb. 16, 2023, which claims priority to Japanese patent application no. 2022-028424, filed on Feb. 25, 2022, the entire contents of which are being incorporated herein by reference.

### BACKGROUND

The present technology relates to an electrolytic solution for a secondary battery, and a secondary battery.

Various kinds of electronic equipment, including mobile phones, have been widely used. Such widespread use has promoted development of a secondary battery as a power source that is smaller in size and lighter in weight and allows for a higher energy density.

The secondary battery includes a positive electrode, a negative electrode, and an electrolytic solution (an electrolytic solution for a secondary battery). A configuration of the secondary battery has been considered in various ways.

Specifically, an electrolytic solution includes an imide compound represented by R_{F}¹—S(═O)₂—NH—S(═O)₂—NH—S(═O)₂—R_{F}².

An electrolyte salt in an electrolytic solution includes an imide anion represented by F—S(═O)₂—N⁻—C(═O)—N⁻—S(═O)₂—F or F—S(═O)₂—N⁻—S(═O)₂—C₆H₄—S(═O)₂—N⁻—S(═O)₂—F.

### SUMMARY

Although consideration has been given in various ways regarding a configuration of a secondary battery, a battery characteristic of the secondary battery is not sufficient yet. Accordingly, there is room for improvement in terms thereof.

It is desirable to provide an electrolytic solution for a secondary battery and a secondary battery each of which makes it possible to achieve a superior battery characteristic.

An electrolytic solution for a secondary battery according to an embodiment of the present technology includes an electrolyte salt and a sulfinyl compound. The electrolyte salt includes an imide anion, and the imide anion includes at least one of an anion represented by Formula (1), an anion represented by Formula (2), an anion represented by Formula (3), or an anion represented by Formula (4). The sulfinyl compound includes at least one of a compound represented by Formula (5), a compound represented by Formula (6), a compound represented by Formula (7), a compound represented by Formula (8), a compound represented by Formula (9), a compound represented by Formula (10), or a compound represented by Formula (11).

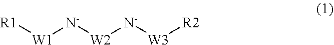

where:
each of R1 and R2 is either a fluorine group or a fluorinated alkyl group; and
each of W1, W2, and W3 is any one of a carbonyl group (>C═O), a sulfinyl group (>S═O), or a sulfonyl group (>S(═O)₂).

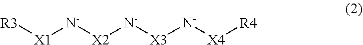

where:
each of R3 and R4 is either a fluorine group or a fluorinated alkyl group; and
each of X1, X2, X3, and X4 is any one of a carbonyl group, a sulfinyl group, or a sulfonyl group.

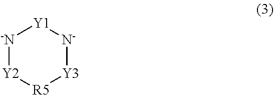

where:
R5 is a fluorinated alkylene group; and
each of Y1, Y2, and Y3 is any one of a carbonyl group, a sulfinyl group, or a sulfonyl group.

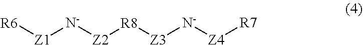

where:
each of R6 and R7 is either a fluorine group or a fluorinated alkyl group;
R8 is any one of an alkylene group, a phenylene group, a fluorinated alkylene group, or a fluorinated phenylene group; and
each of Z1, Z2, Z3, and Z4 is any one of a carbonyl group, a sulfinyl group, or a sulfonyl group.

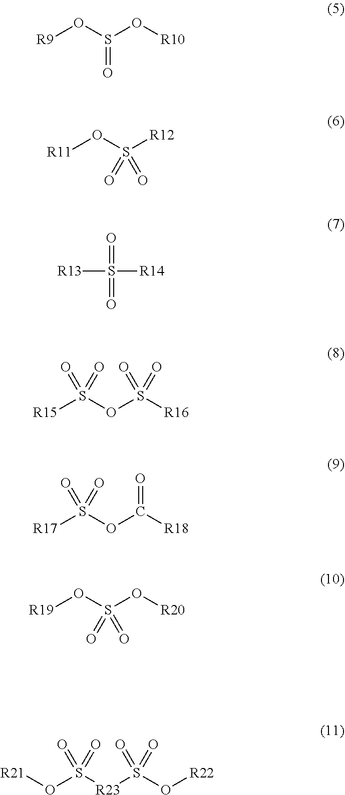

where:
R9 to R22 are either a monovalent hydrocarbon group or a monovalent halogenated hydrocarbon group;
R23 is either a divalent hydrocarbon group or a divalent halogenated hydrocarbon group;
R9 and R10 are optionally bonded to each other;
R11 and R12 are optionally bonded to each other;
R13 and R14 are optionally bonded to each other;
R15 and R16 are optionally bonded to each other;
R17 and R18 are optionally bonded to each other;
R19 and R20 are optionally bonded to each other; and
any two or more of R21 to R23 are optionally bonded to each other.

A secondary battery according to an embodiment of the present technology includes a positive electrode, a negative electrode, and an electrolytic solution. The electrolytic solution has a configuration similar to that of the electrolytic solution for a secondary battery according to the embodiment of the present technology described above.

Note that each of the above-described “monovalent hydrocarbon group”, “monovalent halogenated hydrocarbon group”, “divalent hydrocarbon group”, and “divalent halogenated hydrocarbon group” will be described in detail later.

According to the electrolytic solution for a secondary battery or the secondary battery of an embodiment of the present technology, the electrolytic solution for a secondary battery includes the electrolyte salt and the sulfinyl compound, the electrolyte salt includes at least one of the anion represented by Formula (1), the anion represented by Formula (2), the anion represented by Formula (3), or the anion represented by Formula (4) as the imide anion, and the sulfinyl compound includes at least one of the compound represented by Formula (5), the compound represented by Formula (6), the compound represented by Formula (7), the compound represented by Formula (8), the compound represented by Formula (9), the compound represented by Formula (10), or the compound represented by Formula (11). Accordingly, it is possible to achieve a superior battery characteristic.

Note that effects of the present technology are not necessarily limited to those described above and may include any of a series of effects described below in relation to the present technology.

### BRIEF DESCRIPTION OF THE FIGURES

FIG. 1 is a perspective view of a configuration of a secondary battery according to an embodiment of the present technology.

FIG. 2 is a sectional view of a configuration of a battery device illustrated in FIG. 1.

FIG. 3 is a block diagram illustrating a configuration of an application example of the secondary battery.

### DETAILED DESCRIPTION

The present technology is described below in further detail including with reference to the drawings according to an embodiment.

A description is given first of an electrolytic solution for a secondary battery (hereinafter simply referred to as an “electrolytic solution”) according to an embodiment of the present technology.

The electrolytic solution to be described here is to be used in a secondary battery, which is an electrochemical device. However, the electrolytic solution may be used in other electrochemical devices. The other electrochemical devices are not particularly limited in kind, and specific examples thereof include a capacitor.

The electrolytic solution is a liquid electrolyte, and includes an electrolyte salt and a sulfinyl compound. More specifically, the electrolytic solution includes the electrolyte salt, the sulfinyl compound, and a solvent in which the electrolyte salt and the sulfinyl are each dispersed or dissolved.

The electrolyte salt is a compound that is ionized in the solvent, and includes an anion and a cation. Note that only one kind of electrolyte salt may be used, or two or more kinds of electrolyte salts may be used.

The anion includes an imide anion. The imide anion includes any one or more of an anion represented by Formula (1), an anion represented by Formula (2), an anion represented by Formula (3), or an anion represented by Formula (4). That is, the electrolyte salt includes the imide anion as the anion.

In the following, the anion represented by Formula (1) is referred to as a “first imide anion”, the anion represented by Formula (2) is referred to as a “second imide anion”, the anion represented by Formula (3) is referred to as a “third imide anion”, and the anion represented by Formula (4) is referred to as a “fourth imide anion”.

Note that only one kind of first imide anion may be used, or two or more kinds of first imide anions may be used. That the number of kinds to be used may be one, or two or more as described above is similarly applicable to each of the second imide anion, the third imide anion, and the fourth imide anion.

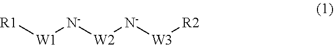

where:
each of R1 and R2 is either a fluorine group or a fluorinated alkyl group; and
each of W1, W2, and W3 is any one of a carbonyl group, a sulfinyl group, or a sulfonyl group.

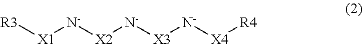

where:
each of R3 and R4 is either a fluorine group or a fluorinated alkyl group; and
each of X1, X2, X3, and X4 is any one of a carbonyl group, a sulfinyl group, or a sulfonyl group.

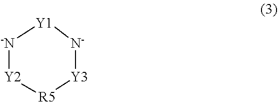

where:
R5 is a fluorinated alkylene group; and
each of Y1, Y2, and Y3 is any one of a carbonyl group, a sulfinyl group, or a sulfonyl group.

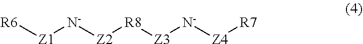

where:
each of R6 and R7 is either a fluorine group or a fluorinated alkyl group;
R8 is any one of an alkylene group, a phenylene group, a fluorinated alkylene group, or a fluorinated phenylene group; and
each of Z1, Z2, Z3, and Z4 is any one of a carbonyl group, a sulfinyl group, or a sulfonyl group.

Reasons why the anion includes the imide anion are as described below. A first reason is that upon charging and discharging of a secondary battery including the electrolytic solution, a high-quality film derived from the electrolyte salt is formed on a surface of each of a positive electrode and a negative electrode. This suppresses a reaction of the electrolytic solution (in particular, the solvent) with each of the positive electrode and the negative electrode, thus suppressing decomposition of the electrolytic solution. A second reason is that, owing to the above-described film, a migration velocity of the cation improves in the vicinity of the surface of each of the positive electrode and the negative electrode. A third reason is that the migration velocity of the cation improves also in the electrolytic solution.

The first imide anion is a chain anion (a divalent negative ion) including two nitrogen atoms (N) and three functional groups (W1 to W3) as represented by Formula (1).

Each of R1 and R2 is not particularly limited as long as each of R1 and R2 is either a fluorine group (—F) or a fluorinated alkyl group. That is, R1 and R2 may be groups that are the same as each other, or may be groups that are different from each other. Accordingly, each of R1 and R2 is neither a hydrogen group (—H) nor an alkyl group.

The fluorinated alkyl group is a group resulting from substituting one or more hydrogen groups (—H) of an alkyl group with one or more fluorine groups. Note that the fluorinated alkyl group may have a straight-chain structure, or may have a branched structure having one or more side chains.

Carbon number of the fluorinated alkyl group is not particularly limited, and is specifically within a range from 1 to 10 both inclusive. A reason for this is that solubility and ionizability of the electrolyte salt including the first imide anion improve.

Specific examples of the fluorinated alkyl group include a perfluoromethyl group (—CF₃) and a perfluoroethyl group (—C₂F₅).

Each of W1 to W3 is not particularly limited as long as each of W1 to W3 is any one of a carbonyl group, a sulfinyl group, or a sulfonyl group. That is, W1 to W3 may be groups that are the same as each other, or may be groups that are different from each other. It goes without saying that only any two of W1 to W3 may be groups that are the same as each other.

The second imide anion is a chain anion (a trivalent negative ion) including three nitrogen atoms and four functional groups (X1 to X4) as represented by Formula (2).

Details of each of R3 and R4 are similar to those of each of R1 and R2.

Each of X1 to X4 is not particularly limited as long as each of X1 to X4 is any one of a carbonyl group, a sulfinyl group, or a sulfonyl group. That is, X1 to X4 may be groups that are the same as each other, or may be groups that are different from each other. It goes without saying that only any two of X1 to X4 may be groups that are the same as each other, and that only any three of X1 to X4 may be groups that are the same as each other.

The third imide anion is a cyclic anion (a divalent negative ion) including two nitrogen atoms, three functional groups (Y1 to Y3), and one linking group (R5) as represented by Formula (3).

The fluorinated alkylene group that is R5 is a group resulting from substituting one or more hydrogen groups of an alkylene group with one or more fluorine groups. Note that the fluorinated alkylene group may have a straight-chain structure, or may have a branched structure having one or more side chains.

Carbon number of the fluorinated alkylene group is not particularly limited, and is specifically within a range from 1 to 10 both inclusive. A reason for this is that solubility and ionizability of the electrolyte salt including the third imide anion improve.

Specific examples of the fluorinated alkylene group include a perfluoromethylene group (—CF₂—) and a perfluoroethylene group (—C₂F₄—).

Each of Y1 to Y3 is not particularly limited as long as each of Y1 to Y3 is any one of a carbonyl group, a sulfinyl group, or a sulfonyl group. That is, Y1 to Y3 may be groups that are the same as each other, or may be groups that are different from each other. It goes without saying that only any two of Y1 to Y3 may be groups that are the same as each other.

The fourth imide anion is a chain anion (a divalent negative ion) including two nitrogen atoms (N), four functional groups (Z1 to Z4), and one linking group (R8) as represented by Formula (4).

Details of each of R6 and R7 are similar to those of each of R1 and R2.

R8 is not particularly limited as long as R8 is any one of an alkylene group, a phenylene group, a fluorinated alkylene group, or a fluorinated phenylene group.

The alkylene group may have a straight-chain structure, or may have a branched structure having one or more side chains. Carbon number of the alkylene group is not particularly limited, and is specifically within a range from 1 to 10 both inclusive. A reason for this is that solubility and ionizability of the electrolyte salt including the fourth imide anion improve. Specific examples of the alkylene group include a methylene group (—CH₂—), an ethylene group (—C₂H₄—), and a propylene group (—C₃H₆—).

Details of the fluorinated alkylene group that is R8 are similar to those of the fluorinated alkylene group that is R5.

The fluorinated phenylene group is a group resulting from substituting one or more hydrogen groups of a phenylene group with one or more fluorine groups. Specific examples of the fluorinated phenylene group include a monofluorophenylene group (—C₆H₃F—).

Each of Z1 to Z4 is not particularly limited as long as each of Z1 to Z4 is any one of a carbonyl group, a sulfinyl group, or a sulfonyl group. That is, Z1 to Z4 may be groups that are the same as each other, or may be groups that are different from each other. It goes without saying that only any two of Z1 to Z4 may be groups that are the same as each other, and that only any three of Z1 to Z4 may be groups that are the same as each other.

Specific examples of the first imide anion include respective anions represented by Formulae (1-1) to (1-30).

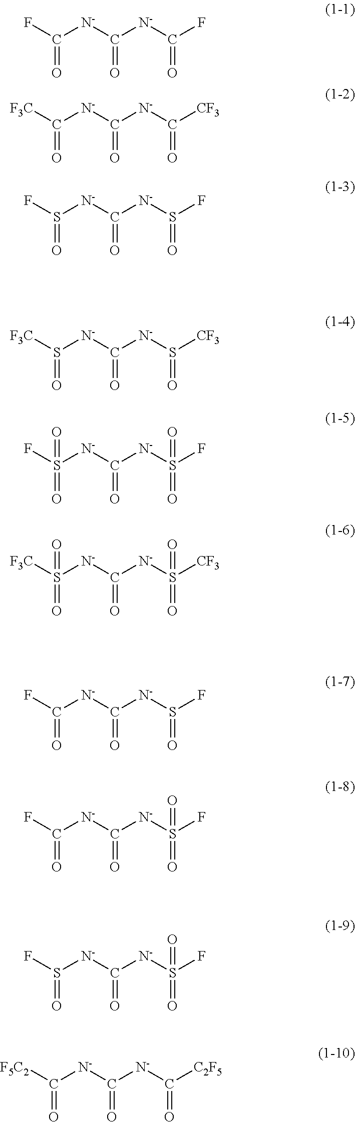

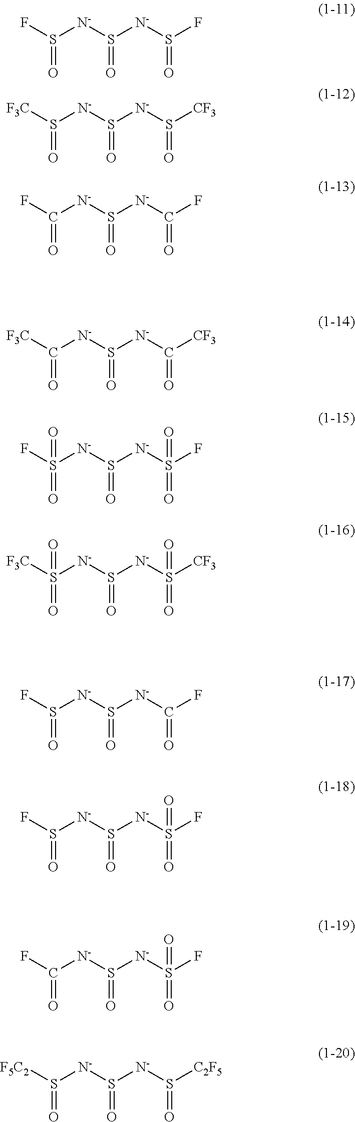

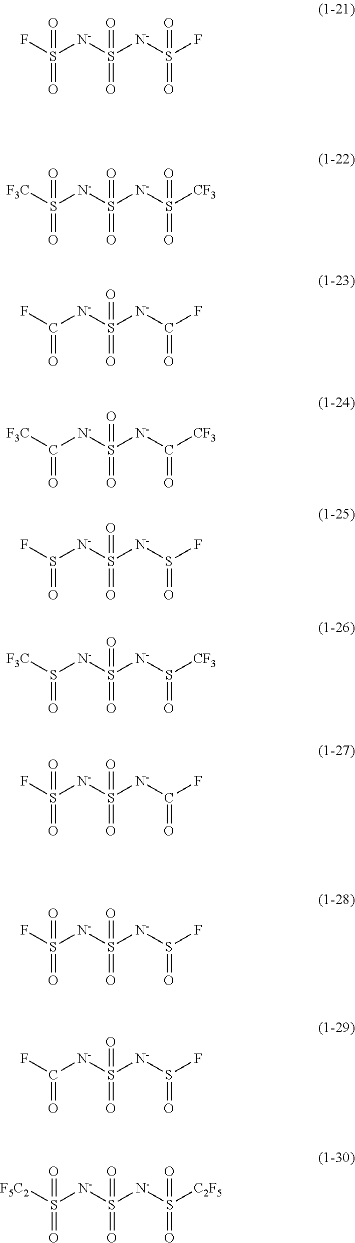

Specific examples of the second imide anion include respective anions represented by Formulae (2-1) to (2-22).

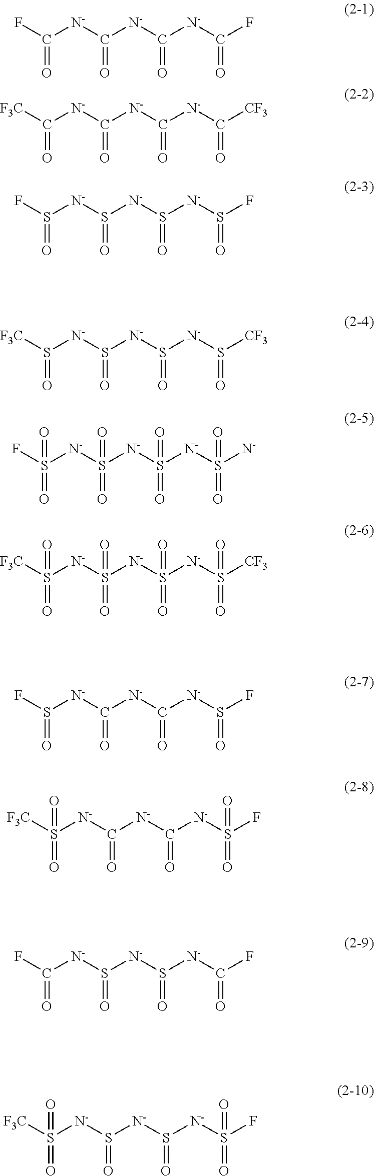

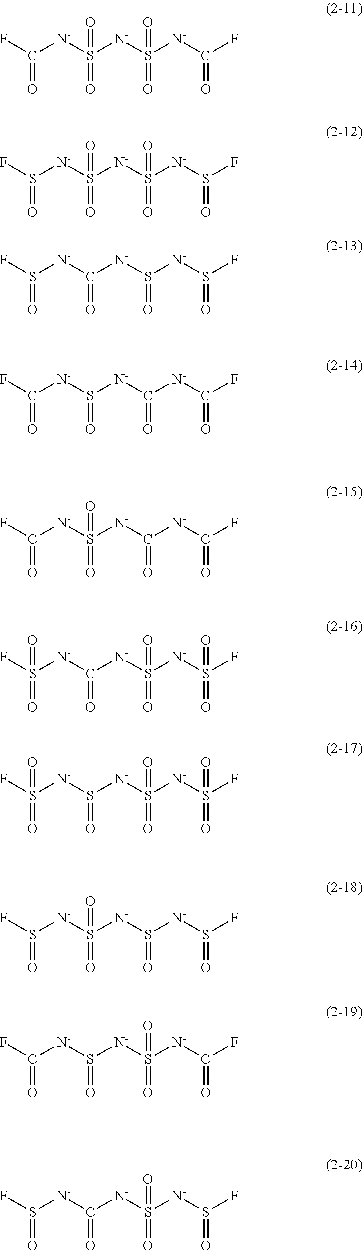

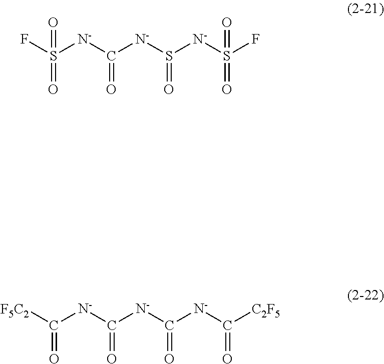

Specific examples of the third imide anion include respective anions represented by Formulae (3-1) to (3-15).

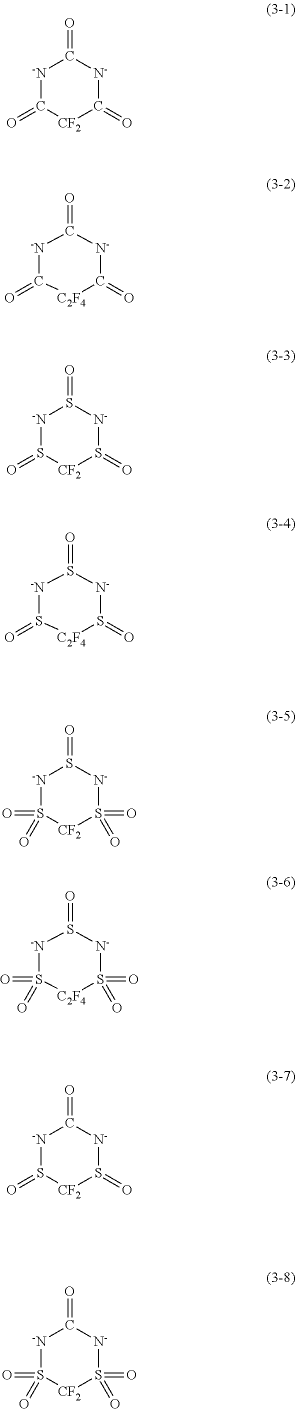

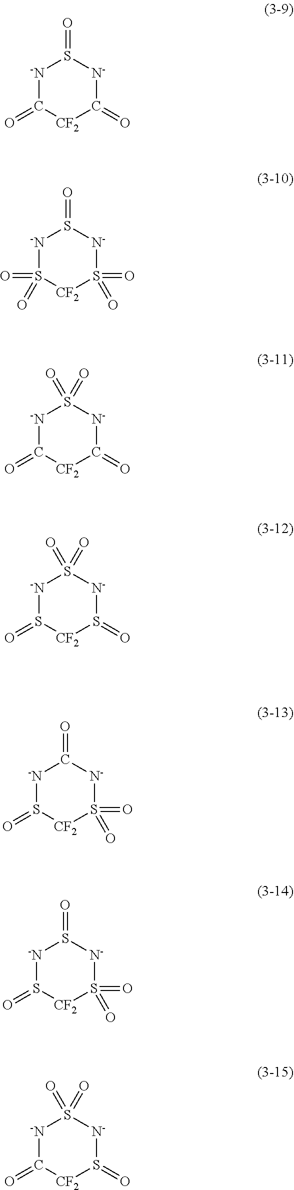

Specific examples of the fourth imide anion include respective anions represented by Formulae (4-1) to (4-65).

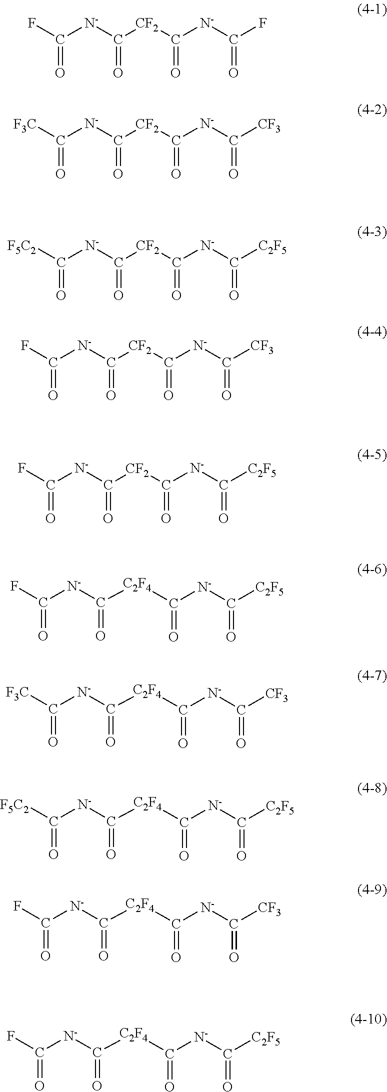

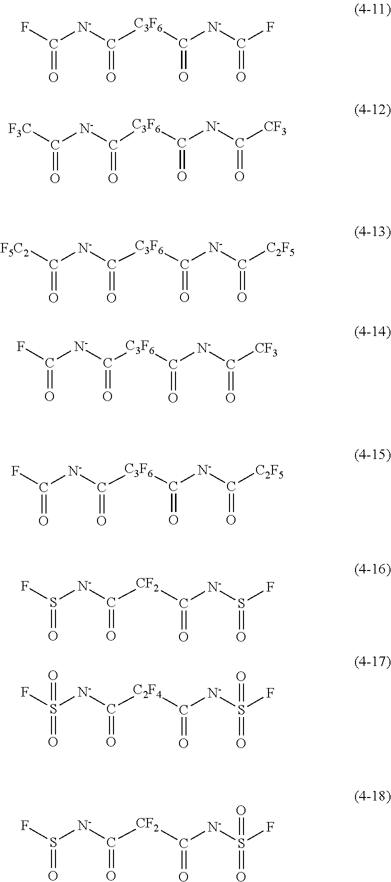

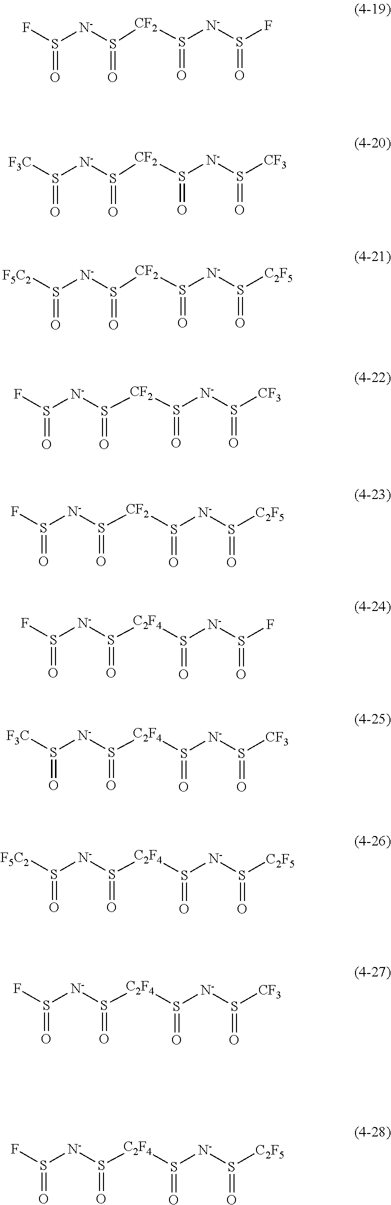

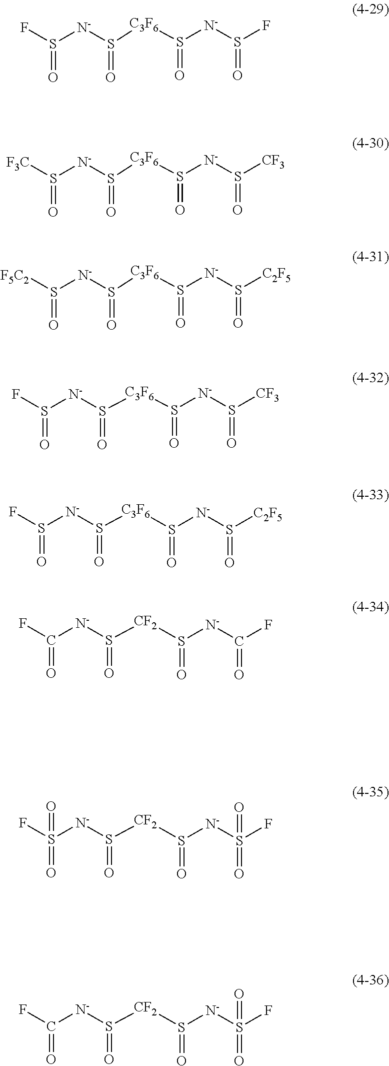

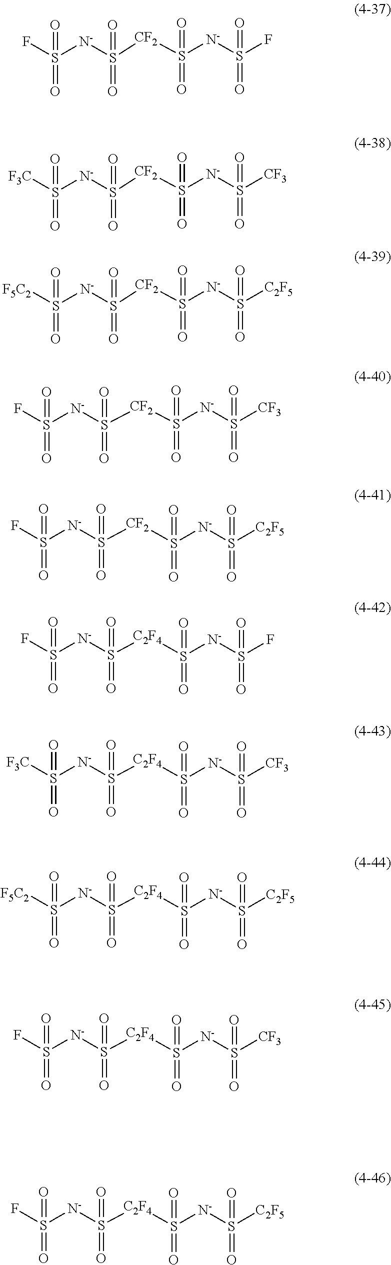

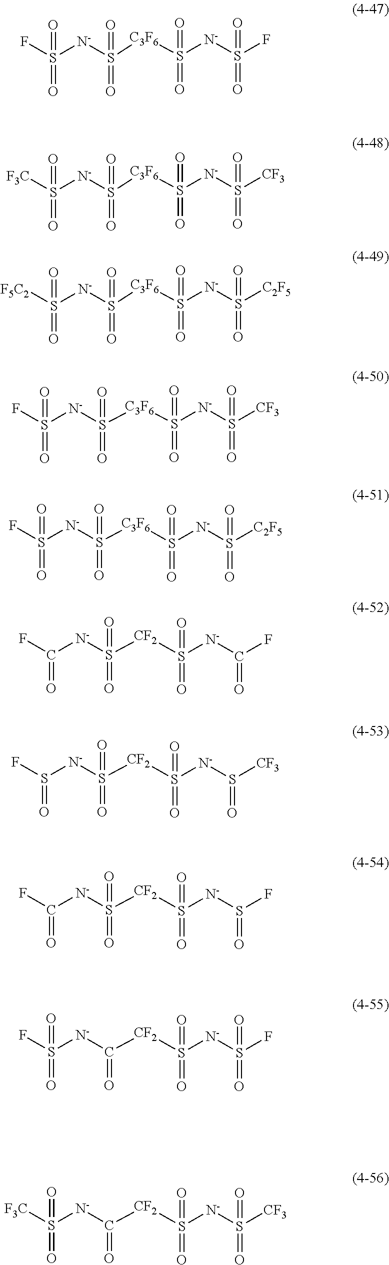

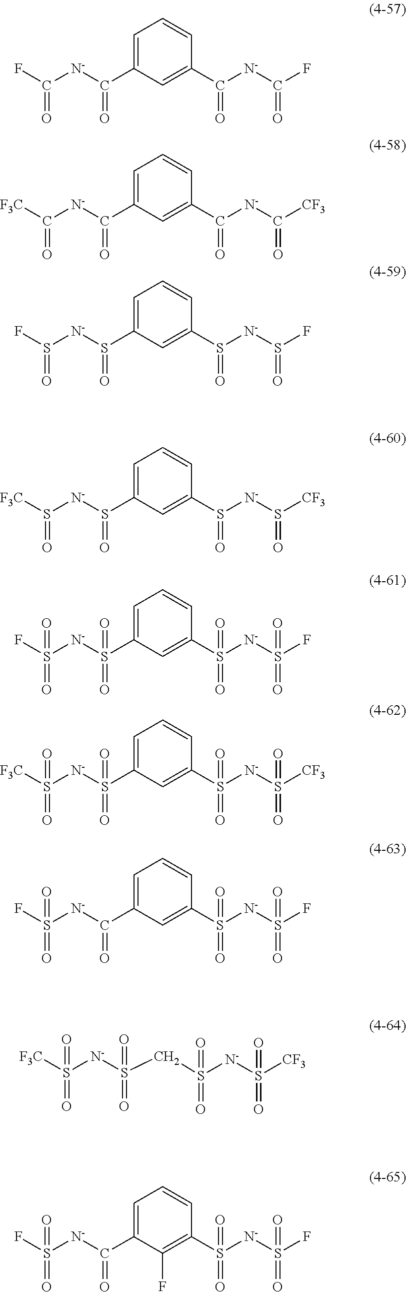

The cation is not particularly limited in kind. Specifically, the cation includes any one or more of light metal ions. That is, the electrolyte salt includes the one or more light metal ions as the cation. A reason for this is that a high voltage is obtainable.

The light metal ion is not particularly limited in kind, and specific examples thereof include an alkali metal ion and an alkaline earth metal ion. Specific examples of the alkali metal ion include a sodium ion and a potassium ion. Specific examples of the alkaline earth metal ion include a beryllium ion, a magnesium ion, and a calcium ion. In addition, the light metal ion may be, for example, an aluminum ion.

In particular, the light metal ion preferably includes a lithium ion. A reason for this is that a sufficiently high voltage is obtainable.

A content of the electrolyte salt in the electrolytic solution is not particularly limited, and may be set as desired. In particular, the content of the electrolyte salt is preferably within a range from 0.2 mol/kg to 2 mol/kg both inclusive. A reason for this is that high ion conductivity is obtainable. The “content of the electrolyte salt” described here refers to the content of the electrolyte salt with respect to the solvent.

In a case of identifying the content of the electrolyte salt, the secondary battery is disassembled to thereby collect the electrolytic solution, following which the electrolytic solution is analyzed by inductively coupled plasma (Inductively Coupled Plasma (ICP)) optical emission spectroscopy. A weight of the solvent and a weight of the electrolyte salt are each thus identified, which allows for a calculation of the content of the electrolyte salt.

The procedure for identifying the content described here is similarly applicable to that in a case of identifying a content of each of components of the electrolytic solution other than the electrolyte salt to be described later. The “components of the electrolytic solution other than the electrolyte salt” include, for example, the sulfinyl compound, any other electrolyte salt, and an additive.

The sulfinyl compound is a compound including one or two sulfinyl groups (—S(═O)—), and more specifically, includes any one or more of a compound represented by Formula (5), a compound represented by Formula (6), a compound represented by Formula (7), a compound represented by Formula (8), a compound represented by Formula (9), a compound represented by Formula (10), or a compound represented by Formula (11). Note that only one kind of sulfinyl compound may be used, or two or more kinds of sulfinyl compounds may be used.

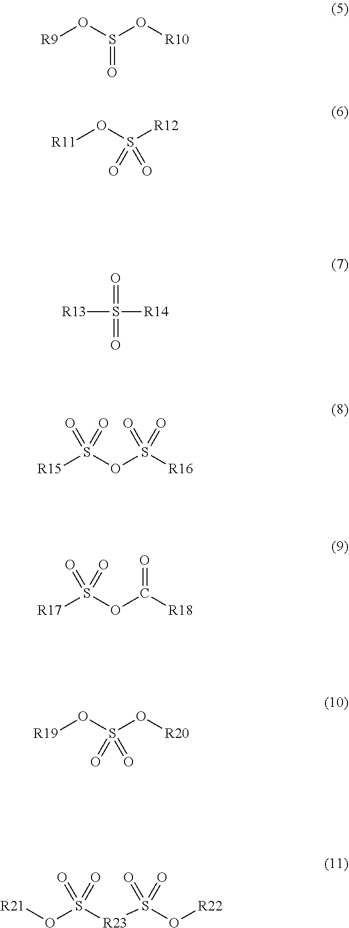

where:
each of R9 to R22 is either a monovalent hydrocarbon group or a monovalent fluorinated hydrocarbon group;
R23 is either a divalent hydrocarbon group or a divalent fluorinated hydrocarbon group;
R9 and R10 are optionally bonded to each other;
R11 and R12 are optionally bonded to each other;
R13 and R14 are optionally bonded to each other;
R15 and R16 are optionally bonded to each other;
R17 and R18 are optionally bonded to each other;
R19 and R20 are optionally bonded to each other; and
any two or more of R21 to R23 are optionally bonded to each other.

A reason why the electrolytic solution includes the sulfinyl compound is that a decomposition reaction of the electrolytic solution is suppressed while ion conductivity is secured. In this case, in particular, the decomposition reaction of the electrolytic solution is effectively suppressed even when the secondary battery including the electrolytic solution is used (charged and discharged) and stored in a high-temperature environment.

The term “monovalent hydrocarbon group” is a generic term for a monovalent group including carbon and hydrogen. The monovalent hydrocarbon group may have a straight-chain structure, or may have a branched structure having one or more side chains. The monovalent hydrocarbon group may include a carbon-carbon unsaturated bond, or may include no carbon-carbon unsaturated bond. The carbon-carbon unsaturated bond may be a carbon-carbon double bond, a carbon-carbon triple bond, or both.

Specific examples of the monovalent hydrocarbon group include an alkyl group, an alkenyl group, an alkynyl group, an aryl group, and a cycloalkyl group.

More specifically, specific examples of the alkyl group include a methyl group (—CH₃), an ethyl group (—C₂H₅), and a propyl group (—C₃H₇). Specific examples of the alkenyl group include a vinyl group (—CH═CH₂) and an allyl group (—CH₂—CH═CH₂). Specific examples of the alkynyl group include an ethynyl group (—C≡CH). Specific examples of the aryl group include a phenyl group and a naphthyl group. Specific examples of the cycloalkyl group include a cyclopropyl group, a cyclobutyl group, a cyclopentyl group, a cyclohexyl group, a cycloheptyl group, and a cyclooctyl group.

Carbon number of the alkyl group is not particularly limited, and is preferably within a range from 1 to 12 both inclusive in particular. Carbon number of each of the alkenyl group and the alkynyl group is not particularly limited, and is preferably within a range from 2 to 12 both inclusive in particular. Carbon number of the aryl group is not particularly limited, and is preferably within a range from 6 to 18 both inclusive in particular. Carbon number of the cycloalkyl group is not particularly limited, and is preferably within a range from 3 to 18 both inclusive in particular. A reason for this is that solubility and compatibility of the sulfinyl compound improve in any of these cases.

The monovalent fluorinated hydrocarbon group is a compound resulting from substituting one or more hydrogen groups of the above-described monovalent hydrocarbon group with one or more fluorine groups. Specific examples of the monovalent fluorinated hydrocarbon group include a perfluoromethyl group (—CF₃) and a perfluoroethyl group (—C₂F₅), which are each a compound resulting from substituting one or more hydrogen groups of an alkyl group with one or more fluorines.

The term “divalent hydrocarbon group” is a generic term for a divalent group including carbon and hydrogen. The divalent hydrocarbon group may have a straight-chain structure, or may have a branched structure having one or more side chains. The divalent hydrocarbon group may include a carbon-carbon unsaturated bond, or may include no carbon-carbon unsaturated bond.

Specific examples of the divalent hydrocarbon group include an alkylene group, an alkenylene group, an alkynylene group, an arylene group, and a cycloalkylene group.

More specifically, specific examples of the alkylene group include a methylene group (—CH₂—), an ethylene group (—C₂H₄—), and a propylene group (—C₃H₆—). Specific examples of the alkenylene group include a vinylene group (—CH═CH—) and an allylene group (—CH₂—CH═CH—). Specific examples of the alkynylene group include an ethynylene group (—C≡C—). Specific examples of the arylene group include a phenylene group and a naphthylene group. Specific examples of the cycloalkylene group include a cyclopropylene group, a cyclobutylene group, a cyclopentylene group, a cyclohexylene group, a cycloheptylene group, and a cyclooctylene group.

Carbon number of the alkylene group is not particularly limited, and is preferably within a range from 1 to 12 both inclusive in particular. Carbon number of each of the alkenylene group and the alkynylene group is not particularly limited, and is preferably within a range from 2 to 12 both inclusive in particular. Carbon number of the arylene group is not particularly limited, and is preferably within a range from 6 to 18 both inclusive in particular. Carbon number of the cycloalkylene group is not particularly limited, and is preferably within a range from 3 to 18 both inclusive in particular. A reason for this is that solubility and compatibility of the sulfinyl compound improve in any of these cases.

The divalent fluorinated hydrocarbon group is a compound resulting from substituting one or more hydrogen groups of the above-described divalent hydrocarbon group with one or more fluorine groups. Specific examples of the divalent fluorinated hydrocarbon group include a perfluoromethylene group (—CF₂—) and a perfluoroethylene group (—C₂F₄—), which are each a compound resulting from substituting one or more hydrogen groups of an alkylene group with one or more fluorines.

Note that Formula (5) represents a state in which R9 and R10 are not bonded to each other, but R9 and R10 may be bonded to each other. Formula (6) represents a state in which R11 and R12 are not bonded to each other, but R11 and R12 may be bonded to each other. Formula (7) represents a state in which R13 and R14 are not bonded to each other, but R13 and R14 may be bonded to each other. Formula (8) represents a state in which R15 and R16 are not bonded to each other, but R15 and R16 may be bonded to each other.

In addition, Formula (9) represents a state in which R17 and R18 are not bonded to each other, but R17 and R18 may be bonded to each other. Formula (10) represents a state in which R19 and R20 are not bonded to each other, but R19 and R20 may be bonded to each other. Formula (11) represents a state in which R21 to R23 are not bonded to each other, but two or more of R21 to R23 may be bonded to each other.

That is, the respective compounds (sulfinyl compounds) represented by Formulae (5) to (11) may include a ring including carbon and hydrogen. The sulfinyl compound may thus be a chain compound including no ring or a cyclic compound including the ring.

Specific examples of the compound represented by Formula (5) include respective compounds represented by Formulae (5-1) to (5-10). Specific examples of the compound represented by Formula (6) include respective compounds represented by Formulae (6-1) to (6-6). Specific examples of the compound represented by Formula (7) include respective compounds represented by Formulae (7-1) to (7-5). Specific examples of the compound represented by Formula (8) include respective compounds represented by Formulae (8-1) to (8-17). Specific examples of the compound represented by Formula (9) include respective compounds represented by Formulae (9-1) to (9-18). Specific examples of the compound represented by Formula (10) include respective compounds represented by Formulae (10-1) to (10-9). Specific examples of the compound represented by Formula (11) include respective compounds represented by Formulae (11-1) to (11-14).

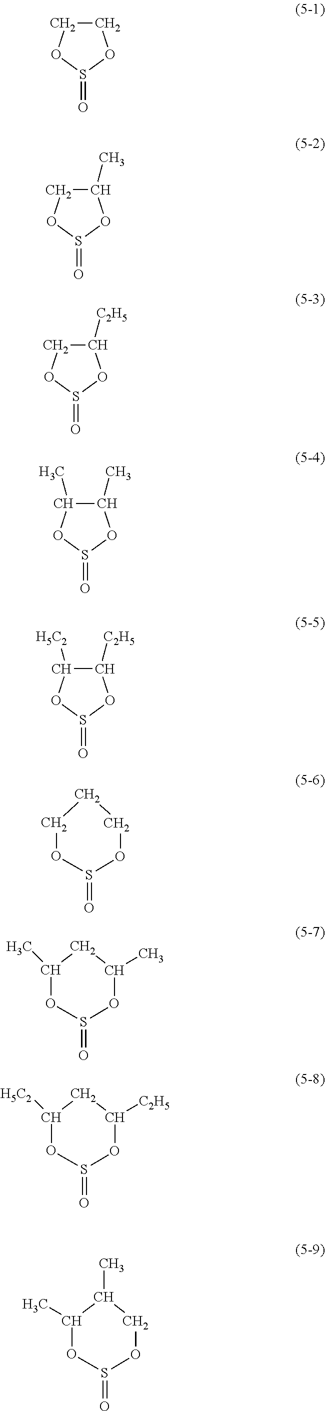

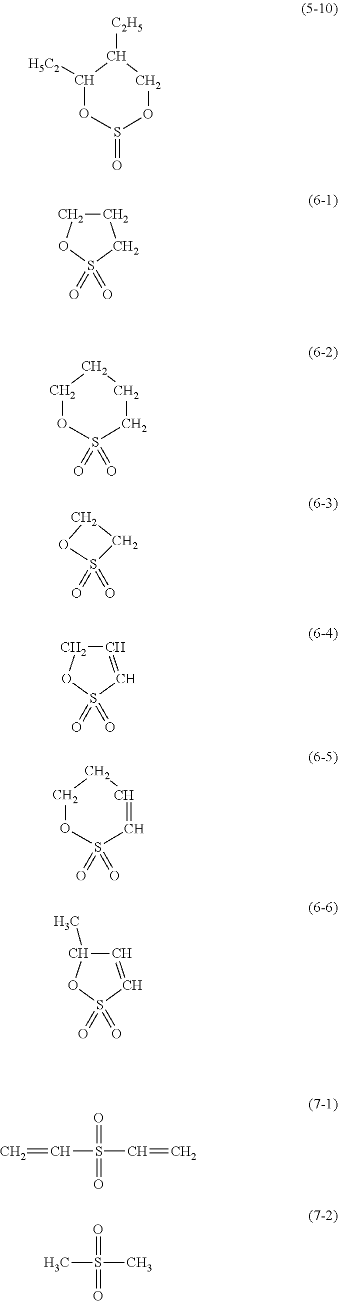

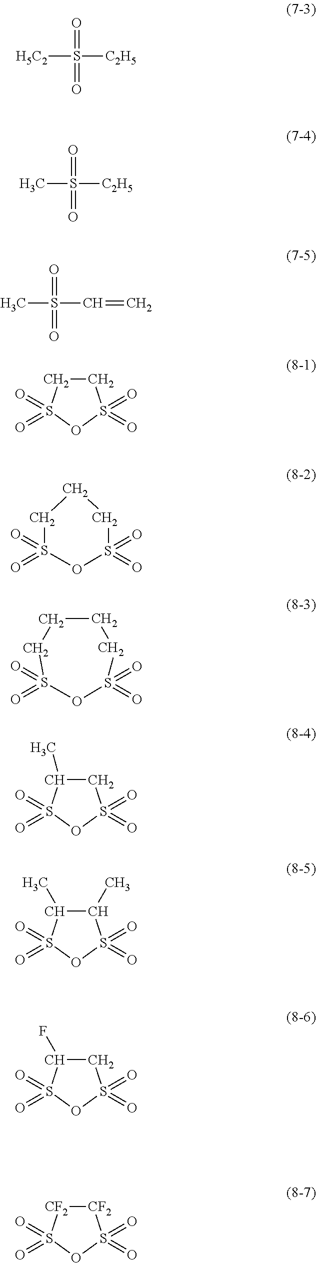

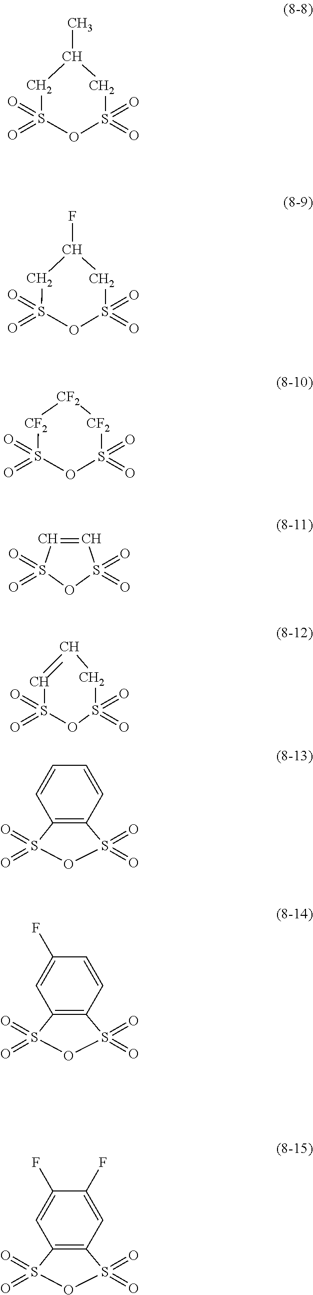

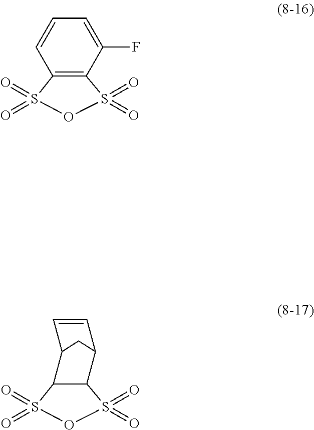 [SMILES: O=S1(=O)OS(=O)(=O)c2c(F)cccc21 ; O=S1(=O)OS(=O)(=O)C2C3C=CC(C3)C21]

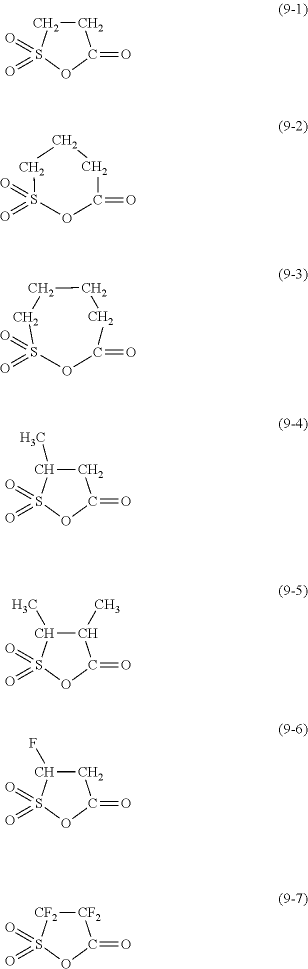

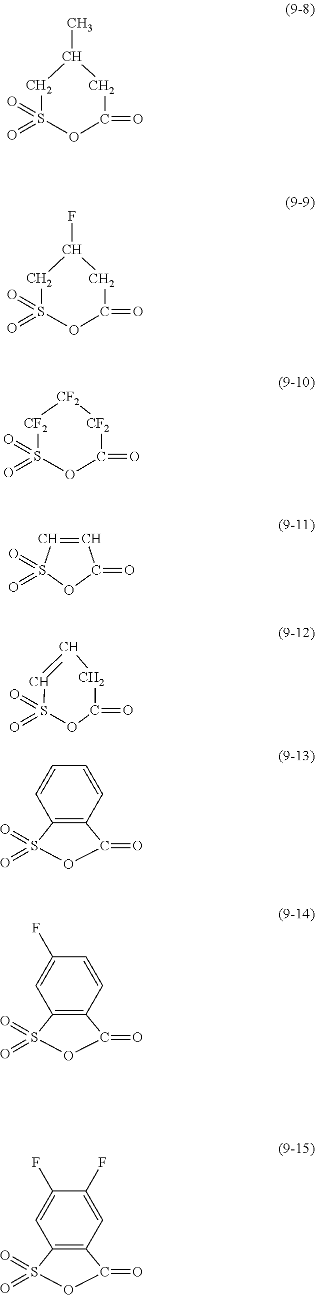

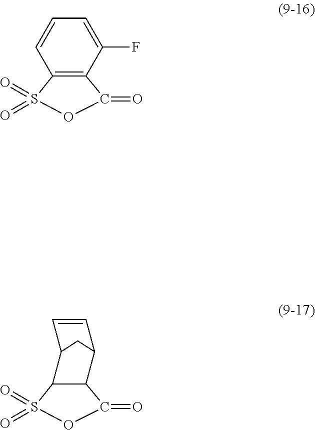 [SMILES: O=C1OS(=O)(=O)c2cccc(F)c21 ; O=C1OS(=O)(=O)C2C3C=CC(C3)C12]

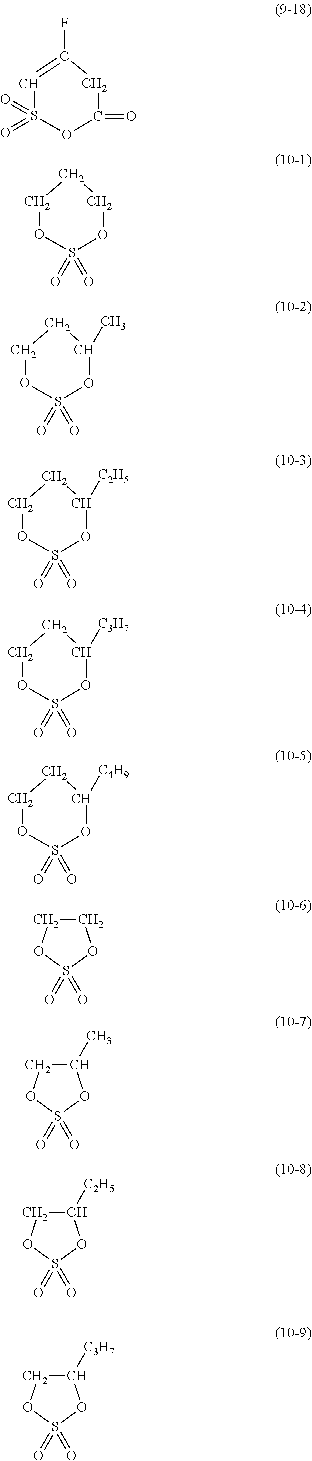

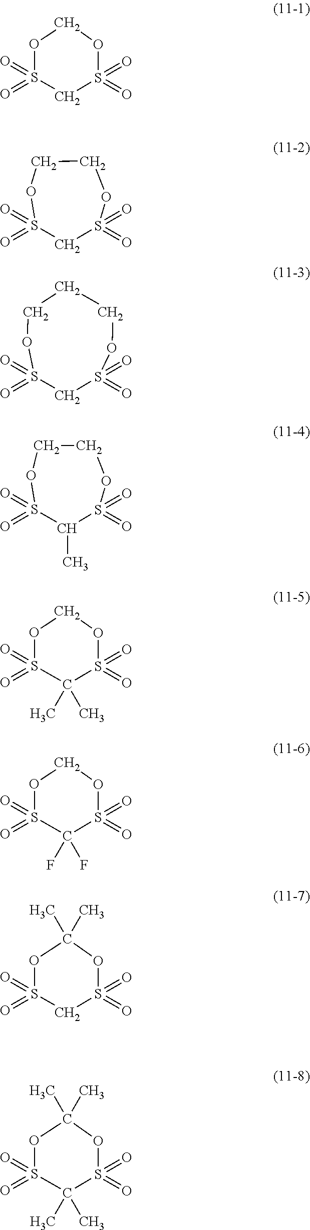

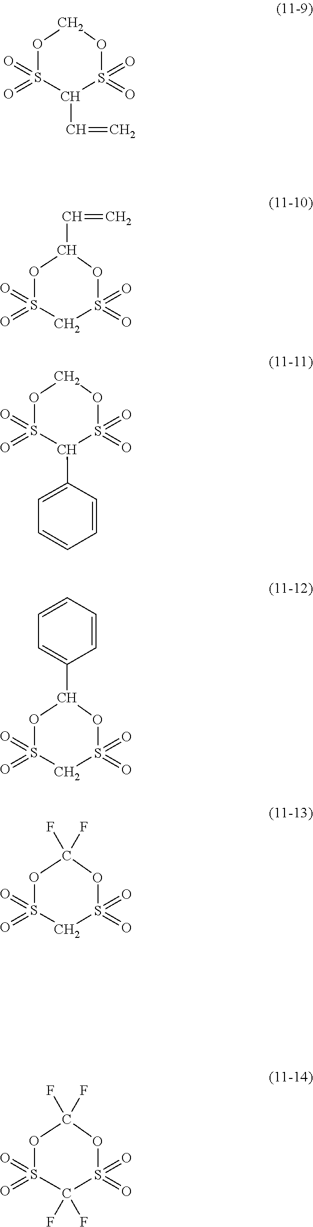 [SMILES: C=CC1S(=O)(=O)OCOS1(=O)=O ; C=CC1OS(=O)(=O)CS(=O)(=O)O1 ; O=S1(=O)OCOS(=O)(=O)C1c1ccccc1 ; O=S1(=O)CS(=O)(=O)OC(c2ccccc2)O1 ; O=S1(=O)CS(=O)(=O)OC(F)(F)O1 ; O=S1(=O)OC(F)(F)OS(=O)(=O)C1(F)F]

A content of the sulfinyl compound in the electrolytic solution is not particularly limited, and may be set as desired. In particular, the content of the sulfinyl compound is preferably within a range from 0.1 wt % to 5 wt % both inclusive. A reason for this is that the decomposition reaction of the electrolytic solution is sufficiently suppressed while ion conductivity is secured.

The solvent includes any one or more of non-aqueous solvents (organic solvents), and the electrolytic solution including the one or more non-aqueous solvents is what is called a non-aqueous electrolytic solution. The non-aqueous solvent is, for example, an ester or an ether, more specifically, a carbonic-acid-ester-based compound, a carboxylic-acid-ester-based compound, or a lactone-based compound, for example.

The carbonic-acid-ester-based compound is, for example, a cyclic carbonic acid ester or a chain carbonic acid ester. Specific examples of the cyclic carbonic acid ester include ethylene carbonate and propylene carbonate. Specific examples of the chain carbonic acid ester include dimethyl carbonate, diethyl carbonate, and ethyl methyl carbonate.

The carboxylic-acid-ester-based compound is, for example, a chain carboxylic acid ester. Specific examples of the chain carboxylic acid ester include methyl acetate, ethyl acetate, methyl propionate, ethyl propionate, propyl propionate, ethyl trimethylacetate, methyl butyrate, and ethyl butyrate.

The lactone-based compound is, for example, a lactone. Specific examples of the lactone include γ-butyrolactone and γ-valerolactone.

Note that the ether may be, for example, 1,2-dimethoxyethane, tetrahydrofuran, 1,3-dioxolane, or 1,4-dioxane.

Note that the electrolytic solution may further include any one or more of other electrolyte salts. A reason for this is that the migration velocity of the cation further improves in the vicinity of the surface of each of the positive electrode and the negative electrode, and the migration velocity of the cation further improves also in the electrolytic solution. A content of the one or more other electrolyte salts in the electrolytic solution is not particularly limited, and may be set as desired.

The one or more other electrolyte salts are not particularly limited in kind, and are each specifically a light metal salt such as a lithium salt. Note that the electrolyte salt described above is excluded from the lithium salt described here.

Specific examples of the lithium salt include lithium hexafluorophosphate (LiPF₆), lithium tetrafluoroborate (LiBF₄), lithium trifluoromethanesulfonate (LiCF₃SO₃), lithium bis(fluorosulfonyl)imide (LiN(FSO₂)₂), lithium bis(trifluoromethanesulfonyl)imide (LiN(CF₃SO₂)₂), lithium tris(trifluoromethanesulfonyl)methide (LiC(CF₃SO₂)₃), lithium bis(oxalato)borate (LiB(C₂O₄)₂), lithium difluorooxalatoborate (LiBF₂(C₂O₄)), lithium difluorodi(oxalato)borate (LiPF₂(C₂O₄)₂), lithium tetrafluorooxalatophosphate (LiPF₄(C₂O₄)), lithium monofluorophosphate (Li₂PFO₃), and lithium difluorophosphate (LiPF₂O₂).

In particular, the one or more other electrolyte salts preferably include any one or more of lithium hexafluorophosphate, lithium tetrafluoroborate, lithium bis(fluorosulfonyl)imide, lithium bis(oxalato)borate, or lithium difluorophosphate. A reason for this is that the migration velocity of the cation sufficiently improves in the vicinity of the surface of each of the positive electrode and the negative electrode, and the migration velocity of the cation sufficiently improves also in the electrolytic solution.

In addition, the electrolytic solution may further include any one or more of additives. A reason for this is that upon charging and discharging of the secondary battery including the electrolytic solution, a film derived from the one or more additives is formed on the surface of each of the positive electrode and the negative electrode, and the decomposition reaction of the electrolytic solution is therefore suppressed. Note that a content of the one or more additives in the electrolytic solution is not particularly limited, and may be set as desired.

The one or more additives are not particularly limited in kind, and specific examples thereof include an unsaturated cyclic carbonic acid ester, a fluorinated cyclic carbonic acid ester, a dicarboxylic acid anhydride, a sulfuric acid ester, a nitrile compound, and an isocyanate compound.

The unsaturated cyclic carbonic acid ester is a cyclic carbonic acid ester having an unsaturated carbon bond (a carbon-carbon double bond). The number of unsaturated carbon bonds is not particularly limited, and may be only one, or two or more. Specific examples of the unsaturated cyclic carbonic acid ester include vinylene carbonate, vinylethylene carbonate, and methylene ethylene carbonate.

The fluorinated cyclic carbonic acid ester is a cyclic carbonic acid ester including fluorine as a constituent element. That is, the fluorinated cyclic carbonic acid ester is a compound resulting from substituting one or more hydrogen groups of a cyclic carbonic acid ester with one or more fluorine groups. Specific examples of the fluorinated cyclic carbonic acid ester include monofluoroethylene carbonate and difluoroethylene carbonate.

Specific examples of the dicarboxylic acid anhydride include succinic anhydride, glutaric anhydride, and maleic anhydride. Specific examples of the sulfuric acid ester include ethylene sulfate (1,3,2-dioxathiolan 2,2-dioxide).

The nitrile compound is a compound including one or more cyano groups (—CN). Specific examples of the nitrile compound include octanenitrile, benzonitrile, phthalonitrile, succinonitrile, glutaronitrile, adiponitrile, sebaconitrile, 1,3,6-hexanetricarbonitrile, 3,3′-oxydipropionitrile, 3-butoxypropionitrile, ethylene glycol bispropionitrile ether, 1,2,2,3-tetracyanopropane, tetracyanopropane, fumaronitrile, 7,7,8,8-tetracyanoquinodimethane, cyclopentanecarbonitrile, 1,3,5-cyclohexanetricarbonitrile, and 1,3-bis(dicyanomethylidene)indane.

The isocyanate compound is a compound including one or more isocyanate groups (—NCO). Specific examples of the isocyanate compound include hexamethylene diisocyanate.

In a case of manufacturing the electrolytic solution, the electrolyte salt is put into the solvent and the solvent is stirred, following which the sulfinyl compound is added to the solvent and the solvent is stirred. In this case, the other electrolyte salt(s) may be further added to the solvent, and the additive(s) may be further added to the solvent. The electrolyte salt and the sulfinyl compound are thereby each dispersed or dissolved in the solvent. As a result, the electrolytic solution is prepared.

According to the electrolytic solution, the electrolytic solution includes the electrolyte salt and the sulfinyl compound, the electrolyte salt includes any one or more of the respective anions represented by Formulae (1) to (4) as the imide anion, and the sulfinyl compound includes any one or more of the respective compounds represented by Formulae (5) to (11).

In this case, as described above, upon charging and discharging of the secondary battery including the electrolytic solution, a high-quality film derived from the electrolyte salt is formed on the surface of each of the positive electrode and the negative electrode, which suppresses the decomposition of the electrolytic solution. In addition, the migration velocity of the cation improves in the vicinity of the surface of each of the positive electrode and the negative electrode, and the migration velocity of the cation improves also in the electrolytic solution.

Further, as described above, the decomposition reaction of the electrolytic solution is suppressed while ion conductivity is secured. In this case, in particular, the decomposition reaction of the electrolytic solution is effectively suppressed even in a high-temperature environment.

Accordingly, in the secondary battery including the electrolytic solution, it is possible to achieve a superior battery characteristic.

In particular, the electrolyte salt may include the light metal ion as the cation. This makes it possible to obtain a high voltage. Accordingly, it is possible to achieve higher effects. In this case, the light metal ion may include a lithium ion. This makes it possible to obtain a higher voltage. Accordingly, it is possible to achieve further higher effects.

Further, the content of the electrolyte salt in the electrolytic solution may be within the range from 0.2 mol/kg to 2 mol/kg both inclusive. This makes it possible to obtain high ion conductivity. Accordingly, it is possible to achieve higher effects.

Further, the content of the sulfinyl compound in the electrolytic solution may be within the range from 0.1 wt % to 5 wt % both inclusive. This sufficiently suppresses the decomposition reaction of the electrolytic solution. Accordingly, it is possible to achieve higher effects.

Further, the electrolytic solution may further include, as the additive(s), any one or more of the unsaturated cyclic carbonic acid ester, the fluorinated cyclic carbonic acid ester, the dicarboxylic acid anhydride, the disulfonic acid anhydride, the sulfuric acid ester, the nitrile compound, or the isocyanate compound. This suppresses the decomposition reaction of the electrolytic solution. Accordingly, it is possible to achieve higher effects.

Further, the electrolytic solution may further include, as the other electrolyte salt(s), any one or more of lithium hexafluorophosphate, lithium tetrafluoroborate, lithium bis(fluorosulfonyl)imide, lithium bis(oxalato)borate, or lithium difluorophosphate. This further improves the migration velocity of the cation. Accordingly, it is possible to achieve higher effects.

A description is given next of a secondary battery including the electrolytic solution described above.

The secondary battery to be described here is a secondary battery in which a battery capacity is obtained through insertion and extraction of an electrode reactant, and includes a positive electrode, a negative electrode, and an electrolytic solution.

In the secondary battery, a charge capacity of the negative electrode is greater than a discharge capacity of the positive electrode. In other words, an electrochemical capacity per unit area of the negative electrode is set to be greater than an electrochemical capacity per unit area of the positive electrode. This is to prevent precipitation of the electrode reactant on a surface of the negative electrode during charging.

The electrode reactant is not particularly limited in kind, and is specifically a light metal such as an alkali metal or an alkaline earth metal. Specific examples of the alkali metal include lithium, sodium, and potassium. Specific examples of the alkaline earth metal include beryllium, magnesium, and calcium. Note that the kind of the electrode reactant may be another light metal such as aluminum.

Examples are given below of a case where the electrode reactant is lithium. A secondary battery in which the battery capacity is obtained through insertion and extraction of lithium is what is called a lithium-ion secondary battery. In the lithium-ion secondary battery, lithium is inserted and extracted in an ionic state.

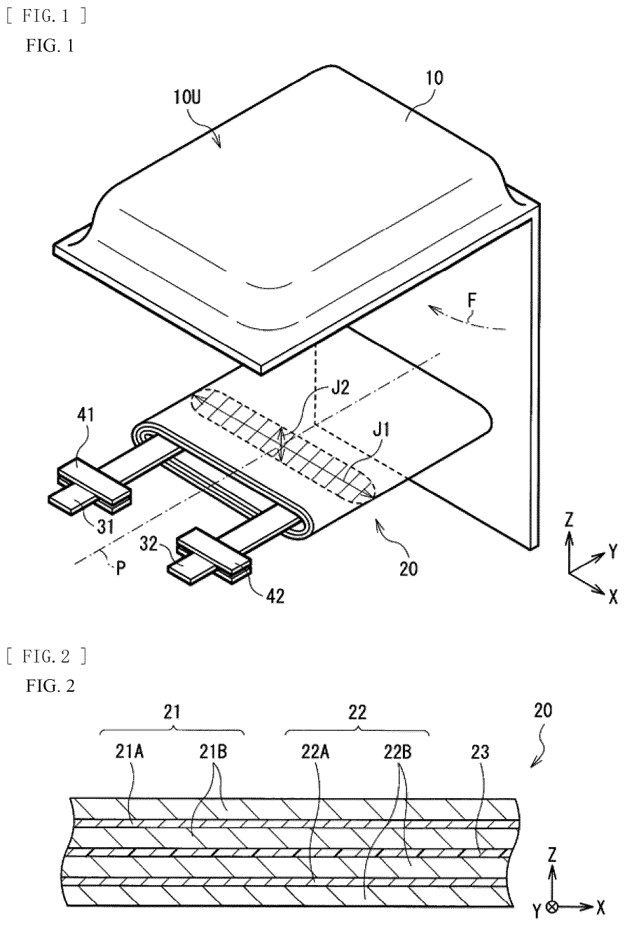

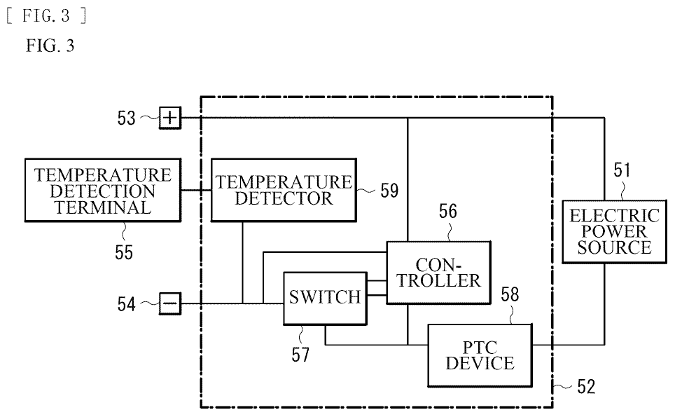

FIG. 1 illustrates a perspective configuration of the secondary battery. FIG. 2 illustrates a sectional configuration of a battery device 20 illustrated in FIG. 1. Note that FIG. 1 illustrates a state in which an outer package film 10 and the battery device 20 are separated from each other, and illustrates a section of the battery device 20 along an XZ plane by a dashed line. FIG. 2 illustrates only a portion of the battery device 20.

As illustrated in FIGS. 1 and 2, the secondary battery includes the outer package film 10, the battery device 20, a positive electrode lead 31, a negative electrode lead 32, and sealing films 41 and 42. The secondary battery described here is a secondary battery of a laminated-film type in which the outer package film 10 having flexibility or softness is used.

As illustrated in FIG. 1, the outer package film 10 is an outer package member that contains the battery device 20. The outer package film 10 has a pouch-shaped structure that is sealed in a state where the battery device 20 is contained inside the outer package film 10. The outer package film 10 thus contains the electrolytic solution together with a positive electrode 21 and a negative electrode 22 that are to be described later.

Here, the outer package film 10 is a single film-shaped member, and is folded in a folding direction F. The outer package film 10 has a depression part 10U to place the battery device 20 therein. The depression part 10U is what is called a deep drawn part.

Specifically, the outer package film 10 is a three-layered laminated film including a fusion-bonding layer, a metal layer, and a surface protective layer that are stacked in this order from an inner side. In a state where the outer package film 10 is folded, outer edge parts of the fusion-bonding layer opposed to each other are fusion-bonded to each other. The fusion-bonding layer includes a polymer compound such as polypropylene. The metal layer includes a metal material such as aluminum. The surface protective layer includes a polymer compound such as nylon.

Note that the outer package film 10 is not particularly limited in configuration or the number of layers, and may be single-layered or two-layered, or may include four or more layers.

The sealing film 41 is interposed between the outer package film 10 and the positive electrode lead 31. The sealing film 42 is interposed between the outer package film 10 and the negative electrode lead 32. Note that the sealing film 41, the sealing film 42, or both may be omitted.

The sealing film 41 is a sealing member that prevents entry, for example, of outside air into the outer package film 10. The sealing film 41 includes a polymer compound such as a polyolefin that has adherence to the positive electrode lead 31. Specific examples of the polyolefin include polypropylene.

A configuration of the sealing film 42 is similar to that of the sealing film 41 except that the sealing film 42 is a sealing member that has adherence to the negative electrode lead 32. That is, the sealing film 42 includes a polymer compound such as a polyolefin that has adherence to the negative electrode lead 32.

As illustrated in FIGS. 1 and 2, the battery device 20 is a power generation device that includes the positive electrode 21, the negative electrode 22, a separator 23, and the electrolytic solution (not illustrated). The battery device 20 is contained inside the outer package film 10.

The battery device 20 is what is called a wound electrode body. That is, in the battery device 20, the positive electrode 21 and the negative electrode 22 are stacked on each other with the separator 23 interposed therebetween, and the positive electrode 21, the negative electrode 22, and the separator 23 are wound about a winding axis P. The winding axis P is a virtual axis extending in a Y-axis direction. The positive electrode 21 and the negative electrode 22 are thus wound, being opposed to each other with the separator 23 interposed therebetween.

A three-dimensional shape of the battery device 20 is not particularly limited. Here, the battery device 20 has an elongated three-dimensional shape. Accordingly, a section of the battery device 20 intersecting the winding axis P, that is, the section of the battery device 20 along the XZ plane, has an elongated shape defined by a major axis J1 and a minor axis J2. The major axis J1 is a virtual axis that extends in an X-axis direction and has a larger length than the minor axis J2. The minor axis J2 is a virtual axis that extends in a Z-axis direction intersecting the X-axis direction and has a smaller length than the major axis J1. Here, the battery device 20 has an elongated cylindrical three-dimensional shape. Thus, the section of the battery device 20 has an elongated, substantially elliptical shape.

The positive electrode 21 includes, as illustrated in FIG. 2, a positive electrode current collector 21A and a positive electrode active material layer 21B.

The positive electrode current collector 21A has two opposed surfaces on each of which the positive electrode active material layer 21B is to be provided. The positive electrode current collector 21A includes an electrically conductive material such as a metal material. Specific examples of the metal material include aluminum.

The positive electrode active material layer 21B includes any one or more of positive electrode active materials into which lithium is insertable and from which lithium is extractable. Note that the positive electrode active material layer 21B may further include any one or more of other materials including, without limitation, a positive electrode binder and a positive electrode conductor.

Here, the positive electrode active material layer 21B is provided on each of the two opposed surfaces of the positive electrode current collector 21A. Note that the positive electrode active material layer 21B may be provided only on one of the two opposed surfaces of the positive electrode current collector 21A on a side where the positive electrode 21 is opposed to the negative electrode 22. A method of forming the positive electrode active material layer 21B is not particularly limited, and specifically includes any one or more of methods including, without limitation, a coating method.

The positive electrode active material is not particularly limited in kind, and specific examples thereof include a lithium-containing compound. The lithium-containing compound is a compound that includes lithium and one or more transition metal elements as constituent elements. The lithium-containing compound may further include one or more other elements as one or more constituent elements. The one or more other elements are not particularly limited in kind as long as the one or more other elements are each an element other than lithium and the transition metal elements. Specifically, the one or more other elements are any one or more of elements belonging to groups 2 to 15 in the long period periodic table. The lithium-containing compound is not particularly limited in kind, and is specifically an oxide, a phosphoric acid compound, a silicic acid compound, or a boric acid compound, for example.

Specific examples of the oxide include LiNiO₂, LiCoO₂, LiCo_{0.98}Al_{0.01}Mg_{0.01}O₂, LiNi_{0.5}Co_{0.2}Mn_{0.3}O₂, LiNi_{0.8}Co_{0.15}Al_{0.05}O₂, LiNi_{0.33}Co_{0.33}Mn_{0.33}O₂, Li_{1.2}Mn_{0.52}Co_{0.175}Ni_{0.1}O₂, Li_{1.15}(Mn_{0.65}Ni_{0.22}Co_{0.13})O₂, and LiMn₂O₄. Specific examples of the phosphoric acid compound include LiFePO₄, LiMnPO₄, LiFe_{0.5}Mn_{0.5}PO₄, and LiFe_{0.3}Mn_{0.7}PO₄.

The positive electrode binder includes any one or more of materials including, without limitation, a synthetic rubber and a polymer compound. Specific examples of the synthetic rubber include a styrene-butadiene-based rubber, a fluorine-based rubber, and ethylene propylene diene. Specific examples of the polymer compound include polyvinylidene difluoride, polyimide, and carboxymethyl cellulose.

The positive electrode conductor includes any one or more of electrically conductive materials including, without limitation, a carbon material. Specific examples of the carbon material include graphite, carbon black, acetylene black, and Ketjen black. Note that the electrically conductive material may be a metal material or a polymer compound, for example.

The negative electrode 22 includes, as illustrated in FIG. 2, a negative electrode current collector 22A and a negative electrode active material layer 22B.

The negative electrode current collector 22A has two opposed surfaces on each of which the negative electrode active material layer 22B is to be provided. The negative electrode current collector 22A includes an electrically conductive material such as a metal material. Specific examples of the metal material include copper.

The negative electrode active material layer 22B includes any one or more of negative electrode active materials into which lithium is insertable and from which lithium is extractable. Note that the negative electrode active material layer 22B may further include any one or more of other materials including, without limitation, a negative electrode binder and a negative electrode conductor.

Here, the negative electrode active material layer 22B is provided on each of the two opposed surfaces of the negative electrode current collector 22A. Note that the negative electrode active material layer 22B may be provided only on one of the two opposed surfaces of the negative electrode current collector 22A on a side where the negative electrode 22 is opposed to the positive electrode 21. A method of forming the negative electrode active material layer 22B is not particularly limited, and specifically includes any one or more of methods including, without limitation, a coating method, a vapor-phase method, a liquid-phase method, a thermal spraying method, and a firing (sintering) method.

The negative electrode active material is not particularly limited in kind, and specific examples thereof include a carbon material, a metal-based material, or both. A reason for this is that a high energy density is obtainable. Specific examples of the carbon material include graphitizable carbon, non-graphitizable carbon, and graphite (natural graphite and artificial graphite). The metal-based material is a material that includes, as one or more constituent elements, any one or more elements among metal elements and metalloid elements that are each able to form an alloy with lithium. Specific examples of such metal elements and metalloid elements include silicon, tin, or both. The metal-based material may be a simple substance, an alloy, a compound, a mixture of two or more thereof, or a material including two or more phases thereof. Specific examples of the metal-based material include TiSi₂ and SiOₓ (0<x≤2 or 0.2<x<1.4).

Details of the negative electrode binder are similar to those of the positive electrode binder. Details of the negative electrode conductor are similar to those of the positive electrode conductor.

As illustrated in FIG. 2, the separator 23 is an insulating porous film interposed between the positive electrode 21 and the negative electrode 22, and allows a lithium ion to pass therethrough while preventing contact (a short circuit) between the positive electrode 21 and the negative electrode 22. The separator 23 includes a polymer compound such as polyethylene.

The positive electrode 21, the negative electrode 22, and the separator 23 are each impregnated with the electrolytic solution, and the electrolytic solution has the configuration described above. That is, the electrolytic solution includes the electrolyte salt and the sulfinyl compound.

As illustrated in FIG. 1, the positive electrode lead 31 is a positive electrode terminal coupled to the positive electrode current collector 21A of the positive electrode 21, and is led from an inside to an outside of the outer package film 10. The positive electrode lead 31 includes an electrically conductive material such as a metal material. Specific examples of the metal material include aluminum. The positive electrode lead 31 is not particularly limited in shape, and specifically has any of shapes including, without limitation, a thin plate shape and a meshed shape.

As illustrated in FIG. 1, the negative electrode lead 32 is a negative electrode terminal coupled to the negative electrode current collector 22A of the negative electrode 22, and is led from the inside to the outside of the outer package film 10. The negative electrode lead 32 includes an electrically conductive material such as a metal material. Specific examples of the metal material include copper. Here, the negative electrode lead 32 is led in a direction similar to that in which the positive electrode lead 31 is led. Note that details of a shape of the negative electrode lead 32 are similar to those of the shape of the positive electrode lead 31.

Upon charging the secondary battery, in the battery device 20, lithium is extracted from the positive electrode 21, and the extracted lithium is inserted into the negative electrode 22 via the electrolytic solution. Upon discharging the secondary battery, in the battery device 20, lithium is extracted from the negative electrode 22, and the extracted lithium is inserted into the positive electrode 21 via the electrolytic solution. Upon charging and discharging, lithium is inserted and extracted in an ionic state.

In a case of manufacturing the secondary battery, the positive electrode 21 and the negative electrode 22 are each fabricated, and the secondary battery is assembled using the positive electrode 21, the negative electrode 22, and the electrolytic solution, following which a stabilization process of the secondary battery is performed, according to an example procedure to be described below. Note that the procedure for preparing the electrolytic solution is as described above.

First, a mixture (a positive electrode mixture) in which the positive electrode active material, the positive electrode binder, and the positive electrode conductor are mixed with each other is put into a solvent to thereby prepare a positive electrode mixture slurry in paste form. The solvent may be an aqueous solvent, or may be an organic solvent. Thereafter, the positive electrode mixture slurry is applied on the two opposed surfaces of the positive electrode current collector 21A to thereby form the positive electrode active material layers 21B. Lastly, the positive electrode active material layers 21B are compression-molded by means of, for example, a roll pressing machine. In this case, the positive electrode active material layers 21B may be heated. The positive electrode active material layers 21B may be compression-molded multiple times. The positive electrode active material layers 21B are thus formed on the two respective opposed surfaces of the positive electrode current collector 21A. As a result, the positive electrode 21 is fabricated.

The negative electrode 22 is formed by a procedure similar to the fabrication procedure of the positive electrode 21 described above. Specifically, first, a mixture (a negative electrode mixture) in which the negative electrode active material, the negative electrode binder, and the negative electrode conductor are mixed with each other is put into a solvent to thereby prepare a negative electrode mixture slurry in paste form. Details of the solvent are as described above. Thereafter, the negative electrode mixture slurry is applied on the two opposed surfaces of the negative electrode current collector 22A to thereby form the negative electrode active material layers 22B. Lastly, the negative electrode active material layers 22B are compression-molded. The negative electrode active material layers 22B are thus formed on the two respective opposed surfaces of the negative electrode current collector 22A. As a result, the negative electrode 22 is fabricated.

First, the positive electrode lead 31 is coupled to the positive electrode current collector 21A of the positive electrode 21 by a coupling method such as a welding method, and the negative electrode lead 32 is coupled to the negative electrode current collector 22A of the negative electrode 22 by a coupling method such as a welding method.

Thereafter, the positive electrode 21 and the negative electrode 22 are stacked on each other with the separator 23 interposed therebetween, following which the stack of the positive electrode 21, the negative electrode 22, and the separator 23 is wound to thereby fabricate a wound body (not illustrated). The wound body has a configuration similar to that of the battery device 20 except that the positive electrode 21, the negative electrode 22, and the separator 23 are each not impregnated with the electrolytic solution. Thereafter, the wound body is pressed by means of, for example, a pressing machine to thereby shape the wound body into an elongated shape.

Thereafter, the wound body is placed inside the depression part 10U, following which the outer package film 10 (the fusion-bonding layer/the metal layer/the surface protective layer) is folded to thereby cause portions of the outer package film 10 to be opposed to each other. Thereafter, outer edge parts of two sides of the fusion-bonding layer opposed to each other are bonded to each other by a bonding method such as a thermal-fusion-bonding method to thereby allow the wound body to be contained inside the outer package film 10 having a pouch shape.

Lastly, the electrolytic solution is injected into the outer package film 10 having the pouch shape, following which outer edge parts of the remaining one side of the fusion-bonding layer opposed to each other are bonded to each other by a bonding method such as a thermal-fusion-bonding method. In this case, the sealing film 41 is interposed between the outer package film 10 and the positive electrode lead 31, and the sealing film 42 is interposed between the outer package film 10 and the negative electrode lead 32.

The wound body is thereby impregnated with the electrolytic solution, and the battery device 20 that is a wound electrode body is thus fabricated. Accordingly, the battery device 20 is sealed in the outer package film 10 having the pouch shape. As a result, the secondary battery is assembled.

The assembled secondary battery is charged and discharged. Various conditions including, for example, an environment temperature, the number of times of charging and discharging (the number of cycles), and charging and discharging conditions may be set as desired. As a result, a film is formed on a surface of each of the positive electrode 21 and the negative electrode 22, which electrochemically stabilizes a state of the secondary battery. As a result, the secondary battery is completed.

According to the secondary battery, the secondary battery includes an electrolytic solution, and the electrolytic solution has the above-described configuration. It is therefore possible to achieve a superior battery characteristic for the reason described above.

In particular, the secondary battery may include a lithium-ion secondary battery. This makes it possible to obtain a sufficient battery capacity stably through insertion and extraction of lithium. Accordingly, it is possible to achieve higher effects.

Other action and effects of the secondary battery are similar to those of the electrolytic solution described above.

The configuration of the secondary battery described above is appropriately modifiable including as described below according to an embodiment. Note that any of the following series of modifications may be combined with each other.

As described above, the electrolytic solution may include, together with the electrolyte salt including the imide anion, the other electrolyte salt(s).

In particular, the electrolytic solution preferably includes lithium hexafluorophosphate as the other electrolyte salt, and the content of the electrolyte salt in the electrolytic solution is preferably made appropriate in relation to a content of lithium hexafluorophosphate in the electrolytic solution.

Specifically, the electrolyte salt includes the cation and the imide anion. The hexafluorophosphate ion includes a lithium ion and a hexafluorophosphate ion.

In this case, a sum T (mol/kg) of a content C1 of the cation in the electrolytic solution and a content C2 of the lithium ion in the electrolytic solution is preferably within a range from 0.7 mol/kg to 2.2 mol/kg both inclusive. Further, a ratio R (mol %) of a number of moles M2 of the hexafluorophosphate ion in the electrolytic solution to a number of moles M1 of the imide anion in the electrolytic solution is preferably within a range from 13 mol % to 6000 mol % both inclusive. A reason for this is that the migration velocity of the cation and a migration velocity of the lithium ion sufficiently improve in the vicinity of the surface of each of the positive electrode 21 and the negative electrode 22, and the migration velocity of the cation and the migration velocity of the lithium ion sufficiently improve also in the electrolytic solution.

The “content of the cation in the electrolytic solution” described above refers to the content of the cation with respect to the solvent, and the “content of the lithium ion in the electrolytic solution” described above refers to the content of the lithium ion with respect to the solvent. Note that the sum T is calculated based on the following calculation expression: T=C1+C2. The ratio R is calculated based on the following calculation expression: R=(M2/M1)×100.

In a case of calculating each of the sum T and the ratio R, the secondary battery is disassembled to thereby collect the electrolytic solution, following which the electrolytic solution is analyzed by ICP optical emission spectroscopy. The content C1, the content C2, the number of moles M1, and the number of moles M2 are each thus identified, which allows for a calculation of each of the sum T and the ratio R.

In this case also, because the electrolytic solution includes the electrolyte salt, it is possible to achieve similar effects. In this case, in particular, when both the electrolyte salt and the other electrolyte salt (lithium hexafluorophosphate) are used, a total amount (the sum T) of the electrolyte salt and the other electrolyte salt is made appropriate, and a mixture ratio (the ratio R) between the electrolyte salt and the other electrolyte salt is also made appropriate. Therefore, the migration velocity of each of the cation and the lithium ion further improves in the vicinity of the surface of each of the positive electrode 21 and the negative electrode 22, and the migration velocity of each of the cation and the lithium ion further improves also in the electrolytic solution. Accordingly, it is possible to achieve higher effects.

The separator 23 that is a porous film is used. However, although not specifically illustrated here, a separator of a stacked type including a polymer compound layer may be used.

Specifically, the separator of the stacked type includes a porous film having two opposed surfaces, and the polymer compound layer provided on one of or each of the two opposed surfaces of the porous film. A reason for this is that adherence of the separator to each of the positive electrode 21 and the negative electrode 22 improves to suppress misalignment (winding displacement) of the battery device 20. This suppresses swelling of the secondary battery even if a side reaction such as the decomposition reaction of the electrolytic solution occurs. The polymer compound layer includes a polymer compound such as polyvinylidene difluoride. A reason for this is that superior physical strength and superior electrochemical stability are obtainable.

Note that the porous film, the polymer compound layer, or both may each include any one or more kinds of insulating particles. A reason for this is that the insulating particles promote heat dissipation upon heat generation by the secondary battery, thus improving safety or heat resistance of the secondary battery. The insulating particles include an inorganic material, a resin material, or both. Specific examples of the inorganic material include aluminum oxide, aluminum nitride, boehmite, silicon oxide, titanium oxide, magnesium oxide, and zirconium oxide. Specific examples of the resin material include acrylic resin and styrene resin.

In a case of fabricating the separator of the stacked type, a precursor solution including, without limitation, the polymer compound and a solvent is prepared, following which the precursor solution is applied on one of or each of the two opposed surfaces of the porous film. In this case, insulating particles may be added to the precursor solution on an as-needed basis.

When the separator of the stacked type is used also, a lithium ion is movable between the positive electrode 21 and the negative electrode 22, and similar effects are therefore obtainable. In this case, in particular, the secondary battery improves in safety, as described above. Accordingly, it is possible to achieve higher effects.

The electrolytic solution, which is a liquid electrolyte, is used. However, although not specifically illustrated here, an electrolyte layer, which is a gel electrolyte, may be used.

In the battery device 20 including the electrolyte layer, the positive electrode 21 and the negative electrode 22 are stacked on each other with the separator 23 and the electrolyte layer interposed therebetween, and the stack of the positive electrode 21, the negative electrode 22, the separator 23, and the electrolyte layer is wound. The electrolyte layer is interposed between the positive electrode 21 and the separator 23, and between the negative electrode 22 and the separator 23.

Specifically, the electrolyte layer includes a polymer compound together with the electrolytic solution. The electrolytic solution is held by the polymer compound. A reason for this is that leakage of the electrolytic solution is prevented. The configuration of the electrolytic solution is as described above. The polymer compound includes, for example, polyvinylidene difluoride. In a case of forming the electrolyte layer, a precursor solution including, for example, the electrolytic solution, the polymer compound, and a solvent is prepared, following which the precursor solution is applied on one side or both sides of the positive electrode 21 and on one side or both sides of the negative electrode 22.

When the electrolyte layer is used also, a lithium ion is movable between the positive electrode 21 and the negative electrode 22 via the electrolyte layer, and similar effects are therefore obtainable. In this case, in particular, the leakage of the electrolytic solution is prevented, as described above. Accordingly, it is possible to achieve higher effects.

Applications (application examples) of the secondary battery are not particularly limited. The secondary battery used as a power source may serve as a main power source or an auxiliary power source of, for example, electronic equipment and an electric vehicle. The main power source is preferentially used regardless of the presence of any other power source. The auxiliary power source is used in place of the main power source, or is switched from the main power source.

Specific examples of the applications of the secondary battery include: electronic equipment; apparatuses for data storage; electric power tools; battery packs to be mounted on, for example, electronic equipment; medical electronic equipment; electric vehicles; and electric power storage systems. Examples of the electronic equipment include video cameras, digital still cameras, mobile phones, laptop personal computers, headphone stereos, portable radios, and portable information terminals. Examples of the apparatuses for data storage include backup power sources and memory cards. Examples of the electric power tools include electric drills and electric saws. Examples of the medical electronic equipment include pacemakers and hearing aids. Examples of the electric vehicles include electric automobiles including hybrid automobiles. Examples of the electric power storage systems include battery systems for home use or industrial use in which electric power is accumulated for a situation such as emergency. In each of the above-described applications, one secondary battery may be used, or multiple secondary batteries may be used.

The battery packs may each include a single battery, or may each include an assembled battery. The electric vehicle is a vehicle that operates (travels) with the secondary battery as a driving power source, and may be a hybrid automobile that is additionally provided with a driving source other than the secondary battery. In the electric power storage system for home use, electric power accumulated in the secondary battery that is an electric power storage source may be utilized for using, for example, home appliances.

An application example of the secondary battery will now be described in detail. The configuration of the application example described below is merely an example, and is appropriately modifiable.

FIG. 3 illustrates a block configuration of a battery pack. The battery pack described here is a battery pack (what is called a soft pack) including one secondary battery, and is to be mounted on, for example, electronic equipment typified by a smartphone.

As illustrated in FIG. 3, the battery pack includes an electric power source 51 and a circuit board 52. The circuit board 52 is coupled to the electric power source 51, and includes a positive electrode terminal 53, a negative electrode terminal 54, and a temperature detection terminal 55.

The electric power source 51 includes one secondary battery. The secondary battery has a positive electrode lead coupled to the positive electrode terminal 53 and a negative electrode lead coupled to the negative electrode terminal 54. The electric power source 51 is couplable to outside via the positive electrode terminal 53 and the negative electrode terminal 54, and is thus chargeable and dischargeable. The circuit board 52 includes a controller 56, a switch 57, a PTC device 58, and a temperature detector 59. However, the PTC device 58 may be omitted.

The controller 56 includes, for example, a central processing unit (CPU) and a memory, and controls an overall operation of the battery pack. The controller 56 detects and controls a use state of the electric power source 51 on an as-needed basis.

If a voltage of the electric power source 51 (the secondary battery) reaches an overcharge detection voltage or an overdischarge detection voltage, the controller 56 turns off the switch 57. This prevents a charging current from flowing into a current path of the electric power source 51. The overcharge detection voltage is not particularly limited, and is specifically 4.20 V±0.05 V. The overdischarge detection voltage is not particularly limited, and is specifically 2.40 V±0.1 V.

The switch 57 includes, for example, a charge control switch, a discharge control switch, a charging diode, and a discharging diode. The switch 57 performs switching between coupling and decoupling between the electric power source 51 and external equipment in accordance with an instruction from the controller 56. The switch 57 includes, for example, a metal-oxide-semiconductor field-effect transistor (MOSFET). The charging and discharging currents are detected based on an ON-resistance of the switch 57.

The temperature detector 59 includes a temperature detection device such as a thermistor. The temperature detector 59 measures a temperature of the electric power source 51 through the temperature detection terminal 55, and outputs a result of the temperature measurement to the controller 56. The result of the temperature measurement to be obtained by the temperature detector 59 is used, for example, when the controller 56 performs charge/discharge control upon abnormal heat generation or when the controller 56 performs a correction process upon calculating a remaining capacity.

### EXAMPLES

A description is given of Examples of the present technology according to an embodiment.

### Examples 1 to 21 and Comparative Examples 1 to 10

Secondary batteries were fabricated, following which the secondary batteries were each evaluated for a battery characteristic as described below.

### [Fabrication of Secondary Battery]

The secondary batteries (lithium-ion secondary batteries) of the laminated-film type illustrated in FIGS. 1 and 2 were fabricated in accordance with the following procedure.

### (Fabrication of Positive Electrode)

First, 91 parts by mass of the positive electrode active material (LiNi_{0.82}Co_{0.14}Al_{0.04}O₂ as the lithium-containing compound (an oxide)), 3 parts by mass of the positive electrode binder (polyvinylidene difluoride), and 6 parts by mass of the positive electrode conductor (carbon black) were mixed with each other to thereby obtain a positive electrode mixture. Thereafter, the positive electrode mixture was put into a solvent (N-methyl-2-pyrrolidone as the organic solvent), following which the solvent was stirred to thereby prepare a positive electrode mixture slurry in paste form. Thereafter, the positive electrode mixture slurry was applied on the two opposed surfaces of the positive electrode current collector 21A (a band-shaped aluminum foil having a thickness of 12 μm) by means of a coating apparatus, following which the applied positive electrode mixture slurry was dried to thereby form the positive electrode active material layers 21B. Lastly, the positive electrode active material layers 21B were compression-molded by means of a roll pressing machine. In this manner, the positive electrode 21 was fabricated.

### (Fabrication of Negative Electrode)

First, 93 parts by mass of the negative electrode active material (artificial graphite as the carbon material, having spacing of a (002) plane of 0.3358 nm measured by X-ray diffractometry) and 7 parts by mass of the negative electrode binder (a styrene-butadiene rubber) were mixed with each other to thereby obtain a negative electrode mixture. Thereafter, the negative electrode mixture was put into a solvent (water as the aqueous solvent), following which the solvent was stirred to thereby prepare a negative electrode mixture slurry in paste form. Thereafter, the negative electrode mixture slurry was applied on the two opposed surfaces of the negative electrode current collector 22A (a band-shaped copper foil having a thickness of 15 μm) by means of a coating apparatus, following which the applied negative electrode mixture slurry was dried to thereby form the negative electrode active material layers 22B. Lastly, the negative electrode active material layers 22B were compression-molded by means of a roll pressing machine. In this manner, the negative electrode 22 was fabricated.

### (Preparation of Electrolytic Solution)

First, the solvent was prepared.

Used as the solvent were ethylene carbonate as the cyclic carbonic acid ester and γ-butyrolactone as the lactone. In this case, a mixture ratio (a weight ratio) between ethylene carbonate and γ-butyrolactone in the solvent was set to 30:70.

Thereafter, the electrolyte salt was put into the solvent, following which the solvent was stirred.

A lithium ion (Li⁺) was used as the cation of the electrolyte salt. Used as the anion of the electrolyte salt were the respective first imide anions represented by Formulae (1-5), (1-6), (1-21), and (1-22), the second imide anion represented by Formula (2-5), the third imide anion represented by Formula (3-5), and the fourth imide anion represented by Formula (4-37). The content (mol/kg) of the electrolyte salt was as listed in Tables 1 and 2.

Lastly, the sulfinyl compound was added to the solvent, following which the solvent was stirred.

Used as the sulfinyl compound were the respective compounds represented by Formulae (5-1), (6-1), (6-4), (7-1), (8-2), (9-1), (10-6), and (11-1). The content (wt %) of the sulfinyl compound was as listed in Tables 1 and 2.

As a result, the electrolytic solution including the electrolyte salt and the sulfinyl compound was prepared. The electrolyte salt was a lithium salt including the imide anion as the anion.

As listed in Table 2, an electrolytic solution for comparison was prepared by a similar procedure, except that a hexafluorophosphate ion (PF₆⁻) was used as the anion instead of the imide anion, and the sulfinyl compound was not used.

In addition, an electrolytic solution for comparison was prepared by a similar procedure, except that a hexafluorophosphate ion was used as the anion instead of the imide anion, and the sulfinyl compound was used.

Further, an electrolytic solution for comparison was prepared by a similar procedure, except that the imide anion was used as the anion, and the sulfinyl compound was not used.

### (Assembly of Secondary Battery)

First, the positive electrode lead 31 (an aluminum foil) was welded to the positive electrode current collector 21A of the positive electrode 21, and the negative electrode lead 32 (a copper foil) was welded to the negative electrode current collector 22A of the negative electrode 22.

Thereafter, the positive electrode 21 and the negative electrode 22 were stacked on each other with the separator 23 (a fine porous polyethylene film having a thickness of 15 μm) interposed therebetween, following which the stack of the positive electrode 21, the negative electrode 22, and the separator 23 was wound to thereby fabricate a wound body. Thereafter, the wound body was pressed by means of a pressing machine, and was thereby shaped into an elongated shape.

Thereafter, the outer package film 10 (the fusion-bonding layer/the metal layer/the surface protective layer) was so folded as to sandwich the wound body placed in the depression part 10U. Thereafter, the outer edge parts of two sides of the fusion-bonding layer were thermal-fusion-bonded to each other to thereby allow the wound body to be contained inside the outer package film 10 having the pouch shape. As the outer package film 10, an aluminum laminated film was used in which the fusion-bonding layer (a polypropylene film having a thickness of 30 μm), the metal layer (an aluminum foil having a thickness of 40 μm), and the surface protective layer (a nylon film having a thickness of 25 μm) were stacked in this order from the inner side.

Lastly, the electrolytic solution was injected into the outer package film 10 having the pouch shape, following which the outer edge parts of the remaining one side of the fusion-bonding layer were thermal-fusion-bonded to each other in a reduced-pressure environment. In this case, the sealing film 41 (a polypropylene film having a thickness of 5 μm) was interposed between the outer package film 10 and the positive electrode lead 31, and the sealing film 42 (a polypropylene film having a thickness of 5 μm) was interposed between the outer package film 10 and the negative electrode lead 32. The wound body was thereby impregnated with the electrolytic solution, and the battery device 20 was thus fabricated.

Accordingly, the battery device was sealed in the outer package film 10. As a result, the secondary battery was assembled.

### (Stabilization of Secondary Battery)

The secondary battery was charged and discharged for one cycle in an ambient temperature environment (at a temperature of 23° C.). Upon charging, the secondary battery was charged with a constant current of 0.1 C until a voltage reached 4.1 V, and was thereafter charged with a constant voltage of that value, 4.1 V, until a current reached 0.05 C. Upon discharging, the secondary battery was discharged with a constant current of 0.1 C until the voltage reached 2.5 V. Note that 0.1 C was a value of a current that caused a battery capacity (a theoretical capacity) to be completely discharged in 10 hours, and 0.05 C was a value of a current that caused the battery capacity to be completely discharged in 20 hours.

A film was thus formed on the surface of each of the positive electrode 21 and the negative electrode 22, and the state of the secondary battery was therefore electrochemically stabilized. As a result, the secondary battery of the laminated-film type was completed.

Note that, after the completion of the secondary battery, the electrolytic solution was analyzed by inductively coupled plasma (Inductively Coupled Plasma (ICP)) optical emission spectroscopy. As a result, it was confirmed that the kind and the content (mol/kg) of the electrolyte salt (the cation and the anion) and the kind and the content (wt %) of the sulfinyl compound were as listed in Tables 1 and 2.

### [Evaluation of Battery Characteristic]

Evaluation of the secondary batteries for their battery characteristics revealed the results presented in Tables 1 and 2. Here, the secondary batteries were each evaluated for a high-temperature cyclability characteristic, a high-temperature storage characteristic, and a low-temperature load characteristic.

### (High-Temperature Cyclability Characteristic)

First, the secondary battery was charged and discharged in a high-temperature environment (at a temperature of 60° C.) to thereby measure a discharge capacity (a first-cycle discharge capacity). Charging and discharging conditions were similar to the charging and discharging conditions for the stabilization of the secondary battery described above.

Thereafter, the secondary battery was repeatedly charged and discharged in the same environment until the total number of cycles reached 100 to thereby measure the discharge capacity (a 100th-cycle discharge capacity). Charging and discharging conditions were similar to the charging and discharging conditions for the stabilization of the secondary battery described above.

Lastly, a cyclability retention rate that was an index for evaluating the high-temperature cyclability characteristic was calculated based on the following calculation expression: cyclability retention rate (%)=(100th-cycle discharge capacity/first-cycle discharge capacity)×100.

### (High-Temperature Storage Characteristic)

First, the secondary battery was charged and discharged for one cycle in an ambient temperature environment (at a temperature of 23° C.) to thereby measure the discharge capacity (a pre-storage discharge capacity). Charging and discharging conditions were similar to the charging and discharging conditions for the stabilization of the secondary battery described above.

Thereafter, the secondary battery was charged in the same environment, and the charged secondary battery was stored (for a storage period of 10 days) in a high-temperature environment (at a temperature of 80° C.). Thereafter, the secondary battery was discharged in the ambient temperature environment to thereby measure the discharge capacity (a post-storage discharge capacity). Charging and discharging conditions were similar to the charging and discharging conditions for the stabilization of the secondary battery described above.

Lastly, a storage retention rate that was an index for evaluating the high-temperature storage characteristic was calculated based on the following calculation expression: storage retention rate (%)=(post-storage discharge capacity/pre-storage discharge capacity)×100.

### (Low-Temperature Load Characteristic)

First, the secondary battery was charged and discharged for one cycle in an ambient temperature environment (at a temperature of 23° C.) to thereby measure the discharge capacity (a first-cycle discharge capacity). Charging and discharging conditions were similar to the charging and discharging conditions for the stabilization of the secondary battery described above.

Thereafter, the secondary battery was repeatedly charged and discharged in a low-temperature environment (at a temperature of −10° C.) until the total number of cycles reached 100 to thereby measure the discharge capacity (a 100th-cycle discharge capacity). Charging and discharging conditions were similar to the charging and discharging conditions for the stabilization of the secondary battery described above, except that the current at the time of discharging was changed to 1 C. Note that 1 C was a value of a current that caused the battery capacity to be completely discharged in 1 hour.

Lastly, a load retention rate that was an index for evaluating the low-temperature load characteristic was calculated based on the following calculation expression: load retention rate (%)=(100th-cycle discharge capacity/first-cycle discharge capacity)×100.

TABLE 1

| | | | | | | Cyclability | Storage | Load |
|---|---|---|---|---|---|---|---|---|
| | Electrolyte salt | | | Sulfinyl compound | | retention | retention | retention |
| | | | Content | | Content | rate | rate | rate |
| | Cation | Anion | (mol/kg) | Kind | (wt %) | (%) | (%) | (%) |
| Example 1 | Li⁺ | Formula (1-21) | 1 | Formula (5-1) | 1 | 85 | 84 | 60 |
| Example 2 | Li⁺ | Formula (1-21) | 1 | Formula (6-1) | 1 | 88 | 90 | 62 |
| Example 3 | Li⁺ | Formula (1-21) | 1 | Formula (6-4) | 1 | 87 | 92 | 60 |
| Example 4 | Li⁺ | Formula (1-21) | 1 | Formula (7-1) | 1 | 82 | 90 | 55 |
| Example 5 | Li⁺ | Formula (1-21) | 1 | Formula (8-2) | 0.1 | 85 | 85 | 60 |
| Example 6 | Li⁺ | Formula (1-21) | 1 | Formula (8-2) | 0.5 | 88 | 88 | 65 |
| Example 7 | Li⁺ | Formula (1-21) | 0.2 | Formula (8-2) | 1 | 55 | 61 | 35 |
| Example 8 | Li⁺ | Formula (1-21) | 0.5 | Formula (8-2) | 1 | 78 | 66 | 48 |
| Example 9 | Li⁺ | Formula (1-21) | 1 | Formula (8-2) | 1 | 90 | 91 | 68 |
| Example 10 | Li⁺ | Formula (1-21) | 2 | Formula (8-2) | 1 | 86 | 87 | 75 |
| Example 11 | Li⁺ | Formula (1-21) | 1 | Formula (8-2) | 2 | 88 | 91 | 64 |
| Example 12 | Li⁺ | Formula (1-21) | 1 | Formula (8-2) | 5 | 82 | 91 | 58 |
| Example 13 | Li⁺ | Formula (1-21) | 1 | Formula (9-1) | 1 | 88 | 90 | 60 |
| Example 14 | Li⁺ | Formula (1-21) | 1 | Formula (10-6) | 1 | 88 | 90 | 65 |
| Example 15 | Li⁺ | Formula (1-21) | 1 | Formula (11-1) | 1 | 88 | 90 | 65 |

TABLE 2

| | | | | | | | Storage | Load |
|---|---|---|---|---|---|---|---|---|
| | Electrolyte salt | | | Sulfinyl compound | | Cyclability | retention | retention |
| | | | Content | | Content | retention | rate | rate |
| | Cation | Anion | (mol/kg) | Kind | (wt %) | rate (%) | (%) | (%) |
| Example 16 | Li⁺ | Formula (1-5) | 1 | Formula (8-2) | 1 | 80 | 82 | 44 |
| Example 17 | Li⁺ | Formula (1-6) | 1 | Formula (8-2) | 1 | 82 | 83 | 44 |
| Example 18 | Li⁺ | Formula (1-22) | 1 | Formula (8-2) | 1 | 86 | 86 | 48 |
| Example 19 | Li⁺ | Formula (2-5) | 1 | Formula (8-2) | 1 | 78 | 80 | 44 |
| Example 20 | Li⁺ | Formula (3-5) | 1 | Formula (8-2) | 1 | 73 | 80 | 42 |
| Example 21 | Li⁺ | Formula (4-37) | 1 | Formula (8-2) | 1 | 50 | 75 | 38 |
| Comparative example 1 | Li⁺ | PF₆⁻ | 1 | — | — | 32 | 60 | 35 |
| Comparative example 2 | Li⁺ | PF₆⁻ | 1 | Formula (5-1) | 1 | 32 | 59 | 35 |
| Comparative example 3 | Li⁺ | PF₆⁻ | 1 | Formula (6-1) | 1 | 35 | 65 | 37 |
| Comparative example 4 | Li⁺ | PF₆⁻ | 1 | Formula (6-4) | 1 | 34 | 65 | 35 |
| Comparative example 5 | Li⁺ | PF₆⁻ | 1 | Formula (7-1) | 1 | 29 | 65 | 40 |
| Comparative example 6 | Li⁺ | PF₆⁻ | 1 | Formula (8-2) | 1 | 37 | 66 | 43 |
| Comparative example 7 | Li⁺ | PF₆⁻ | 1 | Formula (9-1) | 1 | 35 | 65 | 35 |
| Comparative example 8 | Li⁺ | PF₆⁻ | 1 | Formula (10-1) | 1 | 35 | 65 | 40 |
| Comparative example 9 | Li⁺ | PF₆⁻ | 1 | Formula (11-1) | 1 | 35 | 65 | 40 |
| Comparative example 10 | Li⁺ | Formula (1-21) | 1 | — | — | 82 | 80 | 55 |

As indicated in Tables 1 and 2, each of the cyclability retention rate, the storage retention rate, and the load retention rate varied greatly depending on the configuration of the electrolytic solution.

For example, when the electrolyte salt did not include the imide anion and did not include the sulfinyl compound (Comparative example 1), all of the cyclability retention rate, the storage retention rate, and the load retention rate decreased.

In addition, when the electrolyte salt did not include the imide anion but included the sulfinyl compound (Comparative examples 2 to 9), one or more of the cyclability retention rate, the storage retention rate, or the load retention rate increased in some cases, but one or more of the cyclability retention rate, the storage retention rate, or the load retention rate decreased in some cases, as compared with when the electrolytic solution included neither the imide anion nor the sulfinyl compound (Comparative example 1).

Further, when the electrolyte salt included the imide anion but did not include the sulfinyl compound (Comparative example 10), each of the cyclability retention rate, the storage retention rate, and the load retention rate increased, but each of the cyclability retention rate and the storage retention rate did not increase sufficiently, as compared with when the electrolytic solution included neither the imide anion nor the sulfinyl compound (Comparative example 1).

In contrast, when the electrolyte salt included the imide anion and included the sulfinyl compound (Examples 1 to 21), a high cyclability retention rate, a high storage retention rate, and a high load retention rate were obtained. That is, when the electrolytic solution included the imide anion and the sulfinyl compound (Example 9), each of the cyclability retention rate, the storage retention rate, and the load retention rate increased significantly, as compared with when the electrolytic solution included neither the imide anion nor the sulfinyl compound (Comparative example 1).

In this case (Examples 1 to 21), in particular, the following tendencies were obtained. First, when the electrolyte salt included the light metal ion (a lithium ion) as the cation, each of the cyclability retention rate, the storage retention rate, and the load retention rate increased sufficiently. Second, when the content of the electrolyte salt was within the range from 0.2 mol/kg to 2 mol/kg both inclusive with respect to the solvent, each of the cyclability retention rate, the storage retention rate, and the load retention rate increased sufficiently. Third, when the content of the sulfinyl compound was within the range from 0.1 wt % to 5 wt % both inclusive, each of the cyclability retention rate, the storage retention rate, and the load retention rate increased sufficiently.

### Examples 22 to 35

Secondary batteries were fabricated by a procedure similar to that in Example 9, except that either the additive or the other electrolyte salt was added to the electrolytic solution as indicated in Tables 3 and 4, following which the secondary batteries were each evaluated for a battery characteristic.

Details of the additive were as described below. Used as the unsaturated cyclic carbonic acid ester were vinylene carbonate (VC), vinylethylene carbonate (VEC), and methylene ethylene carbonate (MEC). Used as the fluorinated cyclic carbonic acid ester were monofluoroethylene carbonate (FEC) and difluoroethylene carbonate (DFEC). Succinic anhydride (SA) was used as the dicarboxylic acid anhydride. Ethylene sulfate (DTD) was used as the sulfuric acid ester. Succinonitrile (SN) was used as the nitrile compound. Hexamethylene diisocyanate (HMI) was used as the isocyanate compound.

Used as the other electrolyte salt were lithium hexafluorophosphate (LiPF₆), lithium tetrafluoroborate (LiBF₄), lithium bis(fluorosulfonyl)imide (LiFSI), lithium bis(oxalato)borate (LiBOB), and lithium difluorophosphate (LiPF₂O₂).

The content (wt %) of each of the additive and the other electrolyte salt in the electrolytic solution was as listed in Tables 3 and 4. In this case, after the completion of the secondary battery, the electrolytic solution was analyzed by ICP optical emission spectroscopy. As a result, it was confirmed that the content of each of the additive and the other electrolyte salt was as listed in Tables 3 and 4.

TABLE 3

| | | | | | | | | Cyclability | Storage | Load |
|---|---|---|---|---|---|---|---|---|---|---|
| | Electrolyte salt | | | Sulfinyl compound | | Additive | | retention | retention | retention |
| | | | Content | | Content | | Content | rate | rate | rate |
| | Cation | Anion | (mol/kg) | Kind | (wt %) | Kind | (wt %) | (%) | (%) | (%) |
| Example 22 | Li⁺ | Formula (1-21) | 1 | Formula (8-2) | 1 | VC | 1 | 94 | 93 | 66 |
| Example 23 | Li⁺ | Formula (1-21) | 1 | Formula (8-2) | 1 | VEC | 1 | 92 | 93 | 68 |
| Example 24 | Li⁺ | Formula (1-21) | 1 | Formula (8-2) | 1 | MEC | 1 | 92 | 93 | 68 |
| Example 25 | Li⁺ | Formula (1-21) | 1 | Formula (8-2) | 1 | FEC | 5 | 96 | 92 | 68 |
| Example 26 | Li⁺ | Formula (1-21) | 1 | Formula (8-2) | 1 | DFEC | 5 | 93 | 92 | 67 |
| Example 27 | Li⁺ | Formula (1-21) | 1 | Formula (8-2) | 1 | SA | 0.5 | 92 | 93 | 65 |
| Example 28 | Li⁺ | Formula (1-21) | 1 | Formula (8-2) | 1 | DTD | 0.5 | 87 | 89 | 62 |
| Example 29 | Li⁺ | Formula (1-21) | 1 | Formula (8-2) | 1 | SN | 1 | 91 | 94 | 67 |
| Example 30 | Li⁺ | Formula (1-21) | 1 | Formula (8-2) | 1 | HMI | 1 | 91 | 93 | 68 |

TABLE 4

| | | | | | | Other electrolyte | | Cyclability | Storage | Load |
|---|---|---|---|---|---|---|---|---|---|---|
| | Electrolyte salt | | | Sulfinyl compound | | salt | | retention | retention | retention |
| | | | Content | | Content | | Content | rate | rate | rate |
| | Cation | Anion | (mol/kg) | Kind | (wt %) | Kind | (wt %) | (%) | (%) | (%) |
| Example 31 | Li⁺ | Formula (1-21) | 1 | Formula (8-2) | 1 | LiPF₆ | 1 | 67 | 94 | 69 |
| Example 32 | Li⁺ | Formula (1-21) | 1 | Formula (8-2) | 1 | LiBF₄ | 1 | 91 | 93 | 68 |
| Example 33 | Li⁺ | Formula (1-21) | 1 | Formula (8-2) | 1 | LiFSI | 1 | 91 | 94 | 70 |
| Example 34 | Li⁺ | Formula (1-21) | 1 | Formula (8-2) | 1 | LiBOB | 0.5 | 94 | 95 | 66 |
| Example 35 | Li⁺ | Formula (1-21) | 1 | Formula (8-2) | 1 | LiPF₂O₂ | 0.5 | 92 | 93 | 72 |

As indicated in Tables 1 and 3, when the electrolytic solution included the additive (Examples 22 to 30), a high cyclability retention rate, a high storage retention rate, and a high storage retention rate were obtained, as in the case where the electrolytic solution did not include the additive (Example 9). In this case, each of the cyclability retention rate and the storage retention rate further increased while the high load retention rate was secured, depending on the kind of the additive.

Further, as indicated in Tables 1 and 4, when the electrolytic solution included the other electrolyte salt (Examples 31 to 35), a high cyclability retention rate, a high storage retention rate, and a high storage retention rate were obtained, as in the case where the electrolytic solution did not include the other electrolyte salt (Example 9). In this case, each of the cyclability retention rate and the storage retention rate further increased while the high load retention rate was secured, depending on the kind of the other electrolyte salt.

### Examples 36 to 67

Secondary batteries were fabricated by a procedure similar to that in Example 9, except that the other electrolyte salt (lithium hexafluorophosphate (LiPF₆)) was included in the electrolytic solution as indicated in Tables 5 and 6, following which the secondary batteries were each evaluated for a battery characteristic.

In this case, the other electrolyte salt was added to the solvent together with the electrolyte salt, following which the solvent was stirred. The content (mol/kg) of the electrolyte salt, the content (mol/kg) of the other electrolyte salt, the sum T (mol/kg), and the ratio R (mol %) were as listed in Tables 5 and 6.

TABLE 5
Sulfinyl compound = Formula (8-2), Content = 1 wt %

| | | | | Other electrolyte | | | | Cyclability | Storage | Load |
|---|---|---|---|---|---|---|---|---|---|---|
| | Electrolyte salt | | | salt | | | | retention | retention | retention |
| | | | Content | | Content | Sum T | Ratio R | rate | rate | rate |
| | Cation | Anion | (mol/kg) | Kind | (mol/kg) | (mol/kg) | (mol %) | (%) | (%) | (%) |
| Example 36 | Li⁺ | Formula (1-21) | 0.05 | LiPF₆ | 0.1 | 0.15 | 400 | 27 | 46 | 34 |
| Example 37 | Li⁺ | Formula (1-21) | 0.1 | LiPF₆ | 0.1 | 0.2 | 200 | 30 | 58 | 36 |
| Example 38 | Li⁺ | Formula (1-21) | 0.2 | LiPF₆ | 0.1 | 0.3 | 100 | 37 | 63 | 42 |
| Example 39 | Li⁺ | Formula (1-21) | 0.5 | LiPF₆ | 0.1 | 0.6 | 40 | 52 | 73 | 43 |
| Example 40 | Li⁺ | Formula (1-21) | 1 | LiPF₆ | 0.1 | 1.1 | 20 | 92 | 92 | 68 |
| Example 41 | Li⁺ | Formula (1-21) | 1.5 | LiPF₆ | 0.1 | 1.6 | 13 | 95 | 94 | 72 |
| Example 42 | Li⁺ | Formula (1-21) | 2 | LiPF₆ | 0.1 | 2.1 | 10 | 39 | 68 | 45 |
| Example 43 | Li⁺ | Formula (1-21) | 0.05 | LiPF₆ | 0.5 | 0.55 | 2000 | 32 | 63 | 39 |
| Example 44 | Li⁺ | Formula (1-21) | 0.1 | LiPF₆ | 0.5 | 0.6 | 1000 | 35 | 67 | 41 |
| Example 45 | Li⁺ | Formula (1-21) | 0.2 | LiPF₆ | 0.5 | 0.7 | 500 | 62 | 83 | 52 |
| Example 46 | Li⁺ | Formula (1-21) | 0.5 | LiPF₆ | 0.5 | 1 | 200 | 92 | 92 | 69 |
| Example 47 | Li⁺ | Formula (1-21) | 1 | LiPF₆ | 0.5 | 1.5 | 100 | 93 | 93 | 74 |
| Example 48 | Li⁺ | Formula (1-21) | 1.5 | LiPF₆ | 0.5 | 2 | 67 | 72 | 93 | 74 |
| Example 49 | Li⁺ | Formula (1-21) | 0.05 | LiPF₆ | 1 | 1.05 | 4000 | 67 | 76 | 54 |
| Example 50 | Li⁺ | Formula (1-21) | 0.1 | LiPF₆ | 1 | 1.1 | 2000 | 71 | 83 | 60 |
| Example 51 | Li⁺ | Formula (1-21) | 0.2 | LiPF₆ | 1 | 1.2 | 1000 | 82 | 90 | 62 |
| Example 52 | Li⁺ | Formula (1-21) | 0.5 | LiPF₆ | 1 | 1.5 | 400 | 92 | 94 | 64 |

TABLE 6
Sulfinyl compound = Formula (8-2), Content = 1 wt %

| | | | | Other electrolyte | | | | Cyclability | Storage | Load |
|---|---|---|---|---|---|---|---|---|---|---|
| | Electrolyte salt | | | salt | | | | retention | retention | retention |
| | | | Content | | Content | Sum T | Ratio R | rate | rate | rate |
| | Cation | Anion | (mol/kg) | Kind | (mol/kg) | (mol/kg) | (mol %) | (%) | (%) | (%) |
| Example 53 | Li⁺ | Formula (1-21) | 1 | LiPF₆ | 1 | 2 | 200 | 67 | 94 | 69 |
| Example 54 | Li⁺ | Formula (1-21) | 1.5 | LiPF₆ | 1 | 2.5 | 133 | 35 | 73 | 50 |
| Example 55 | Li⁺ | Formula (1-21) | 0.05 | LiPF₆ | 1.2 | 1.25 | 4800 | 67 | 83 | 56 |
| Example 56 | Li⁺ | Formula (1-21) | 0.1 | LiPF₆ | 1.2 | 1.3 | 2400 | 72 | 88 | 62 |
| Example 57 | Li⁺ | Formula (1-21) | 0.2 | LiPF₆ | 1.2 | 1.4 | 1200 | 82 | 91 | 66 |
| Example 58 | Li⁺ | Formula (1-21) | 0.5 | LiPF₆ | 1.2 | 1.7 | 480 | 92 | 94 | 69 |
| Example 59 | Li⁺ | Formula (1-21) | 1 | LiPF₆ | 1.2 | 2.2 | 240 | 65 | 94 | 72 |
| Example 60 | Li⁺ | Formula (1-21) | 1.5 | LiPF₆ | 1.2 | 2.7 | 160 | 32 | 78 | 50 |
| Example 61 | Li⁺ | Formula (1-21) | 0.05 | LiPF₆ | 1.5 | 1.55 | 6000 | 62 | 80 | 59 |
| Example 62 | Li⁺ | Formula (1-21) | 0.1 | LiPF₆ | 1.5 | 1.6 | 3000 | 65 | 83 | 64 |
| Example 63 | Li⁺ | Formula (1-21) | 0.2 | LiPF₆ | 1.5 | 1.7 | 1500 | 69 | 82 | 69 |
| Example 64 | Li⁺ | Formula (1-21) | 0.5 | LiPF₆ | 1.5 | 2 | 600 | 62 | 94 | 72 |
| Example 65 | Li⁺ | Formula (1-21) | 1 | LiPF₆ | 1.5 | 2.5 | 300 | 32 | 74 | 50 |
| Example 66 | Li⁺ | Formula (1-21) | 1.5 | LiPF₆ | 1.5 | 3 | 200 | 22 | 71 | 46 |
| Example 67 | Li⁺ | Formula (1-21) | 0.05 | LiPF₆ | 2 | 2.05 | 8000 | 32 | 68 | 45 |

As indicated in Tables 5 and 6, when two conditions, i.e., a condition that the sum T was within the range from 0.7 mol/kg to 2.2 mol/kg both inclusive and a condition that the ratio R was within the range from 13 mol % to 6000 mol % both inclusive, were satisfied (Example 40, etc.), each of the cyclability retention rate, the storage retention rate, and the load retention rate further increased, as compared with when at least one of the two conditions was not satisfied (Example 36, etc.).

Based upon the results presented in Tables 1 to 6, when: the electrolytic solution included the electrolyte salt and the sulfinyl compound; the electrolyte salt included any one or more of the respective anions represented by Formulae (1) to (4) as the imide anion; and the sulfinyl compound included any one or more of the respective compounds represented by Formulae (5) to (11), all of the cyclability retention rate, the storage retention rate, and the load retention rate improved. Therefore, a superior high-temperature cyclability characteristic, a superior high-temperature storage characteristic, and a superior low-temperature load characteristic of the secondary battery were achieved. Accordingly, it was possible to achieve a superior battery characteristic.

Although the present technology has been described above with reference to some embodiments and Examples, the configuration of the present technology is not limited to those described with reference to the embodiments and Examples above, and is therefore modifiable in a variety of ways.

For example, the description has been given of the case where the battery device has a device structure of a wound type. However, the device structure of the battery device is not particularly limited. Therefore, the device structure may be, for example, a stacked type or a zigzag folded type. In the stacked type, the positive electrode and the negative electrode are alternately stacked on each other with the separator interposed therebetween. In the zigzag folded type, the positive electrode and the negative electrode are opposed to each other with the separator interposed therebetween, and are folded in a zigzag manner.

Further, although the description has been given of the case where the electrode reactant is lithium, the electrode reactant is not particularly limited. Specifically, the electrode reactant may be another alkali metal such as sodium or potassium, or may be an alkaline earth metal such as beryllium, magnesium, or calcium, as described above. In addition, the electrode reactant may be another light metal such as aluminum.

The effects described herein are mere examples, and effects of the present technology are therefore not limited to those described herein. Accordingly, the present technology may achieve any other effect.

It should be understood that various changes and modifications to the embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A secondary battery comprising:
a positive electrode;
a negative electrode; and
an electrolytic solution including an electrolyte salt and a sulfinyl compound, wherein
the electrolyte salt includes an imide anion, and the imide anion includes at least one of an anion represented by Formula (1), an anion represented by Formula (2), an anion represented by Formula (3), or an anion represented by Formula (4), and
the sulfinyl compound includes at least one of a compound represented by Formula (5), a compound represented by Formula (6), a compound represented by Formula (7), a compound represented by Formula (8), a compound represented by Formula (9), a compound represented by Formula (10), or a compound represented by Formula (11), 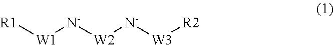
where
each of R1 and R2 is either a fluorine group or a fluorinated alkyl group, and
each of W1, W2, and W3 is any one of a carbonyl group (>C═O), a sulfinyl group (>S═O), or a sulfonyl group (>S(═O)₂), 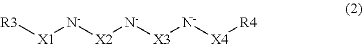
where
each of R3 and R4 is either a fluorine group or a fluorinated alkyl group, and
each of X1, X2, X3, and X4 is any one of a carbonyl group, a sulfinyl group, or a sulfonyl group, 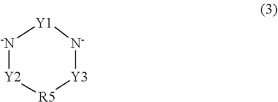
where
R5 is a fluorinated alkylene group, and
each of Y1, Y2, and Y3 is any one of a carbonyl group, a sulfinyl group, or a sulfonyl group, 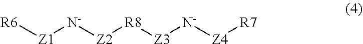
where
each of R6 and R7 is either a fluorine group or a fluorinated alkyl group,
R8 is any one of an alkylene group, a phenylene group, a fluorinated alkylene group, or a fluorinated phenylene group, and
each of Z1, Z2, Z3, and Z4 is any one of a carbonyl group, a sulfinyl group, or a sulfonyl group, 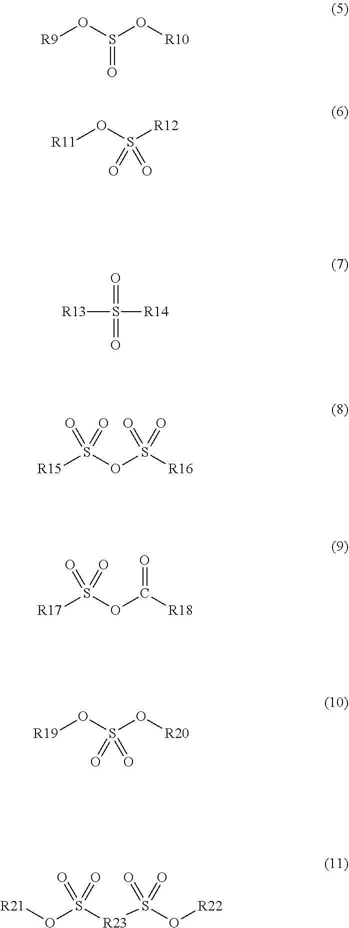
where
each of R9 to R22 is either a monovalent hydrocarbon group or a monovalent fluorinated hydrocarbon group,
R23 is either a divalent hydrocarbon group or a divalent fluorinated hydrocarbon group,
R9 and R10 are optionally bonded to each other,
R11 and R12 are optionally bonded to each other,
R13 and R14 are optionally bonded to each other,
R15 and R16 are optionally bonded to each other,
R17 and R18 are optionally bonded to each other,
R19 and R20 are optionally bonded to each other, and
any two or more of R21 to R23 are optionally bonded to each other.

2. The secondary battery according to claim 1, wherein
the electrolyte salt further includes a cation, and
the cation includes a light metal ion.

3. The secondary battery according to claim 2, wherein the light metal ion includes a lithium ion.

4. The secondary battery according to claim 1, wherein a content of the electrolyte salt in the electrolytic solution is greater than or equal to 0.2 moles per kilogram and less than or equal to 2 moles per kilogram.

5. The secondary battery according to claim 1, wherein
the electrolytic solution further includes lithium hexafluorophosphate,
the electrolyte salt includes a cation and the imide anion,
the lithium hexafluorophosphate includes a lithium ion and a hexafluorophosphate ion,
a sum of a content of the cation in the electrolytic solution and a content of the lithium ion in the electrolytic solution is greater than or equal to 0.7 moles per kilogram and less than or equal to 2.2 moles per kilogram, and
a ratio of a number of moles of the hexafluorophosphate ion in the electrolytic solution to a number of moles of the imide anion in the electrolytic solution is greater than or equal to 13 mole percent and less than or equal to 6000 mole percent.

6. The secondary battery according to claim 1, wherein a content of the sulfinyl compound in the electrolytic solution is greater than or equal to 0.1 weight percent and less than or equal to 5 weight percent.

7. The secondary battery according to claim 1, wherein the electrolytic solution further includes at least one of an unsaturated cyclic carbonic acid ester, a fluorinated cyclic carbonic acid ester, a dicarboxylic acid anhydride, a sulfuric acid ester, a nitrile compound, or an isocyanate compound.

8. The secondary battery according to claim 1, wherein the electrolytic solution further includes at least one of lithium hexafluorophosphate, lithium tetrafluoroborate, lithium bis(fluorosulfonyl)imide, lithium bis(oxalato)borate, or lithium difluorophosphate.

9. The secondary battery according to claim 1, wherein the secondary battery comprises a lithium-ion secondary battery.

10. An electrolytic solution for a secondary battery, the electrolytic solution comprising:
an electrolyte salt; and
a sulfinyl compound, wherein
the electrolyte salt includes an imide anion, and the imide anion includes at least one of an anion represented by Formula (1), an anion represented by Formula (2), an anion represented by Formula (3), or an anion represented by Formula (4), and
the sulfinyl compound includes at least one of a compound represented by Formula (5), a compound represented by Formula (6), a compound represented by Formula (7), a compound represented by Formula (8), a compound represented by Formula (9), a compound represented by Formula (10), or a compound represented by Formula (11), 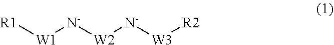
where
each of R1 and R2 is either a fluorine group or a fluorinated alkyl group, and
each of W1, W2, and W3 is any one of a carbonyl group, a sulfinyl group, or a sulfonyl group, 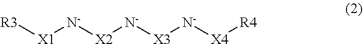
where
each of R3 and R4 is either a fluorine group or a fluorinated alkyl group, and
each of X1, X2, X3, and X4 is any one of a carbonyl group, a sulfinyl group, or a sulfonyl group, 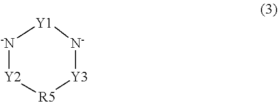
where
R5 is a fluorinated alkylene group, and
each of Y1, Y2, and Y3 is any one of a carbonyl group, a sulfinyl group, or a sulfonyl group, 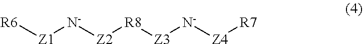
where
each of R6 and R7 is either a fluorine group or a fluorinated alkyl group,
R8 is any one of an alkylene group, a phenylene group, a fluorinated alkylene group, or a fluorinated phenylene group, and
each of Z1, Z2, Z3, and Z4 is any one of a carbonyl group, a sulfinyl group, or a sulfonyl group, 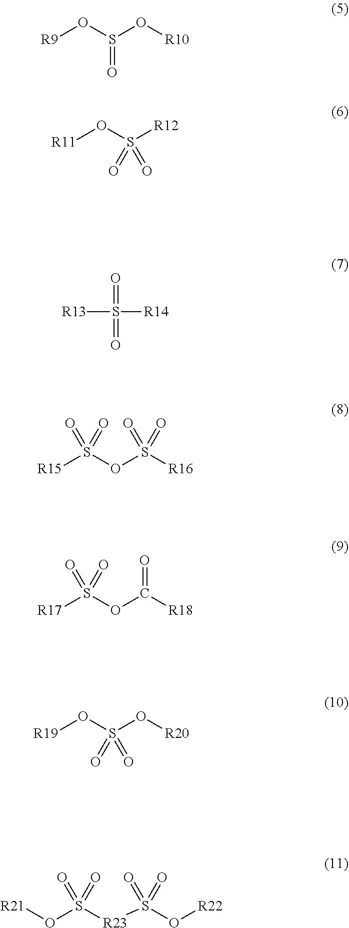
where
each of R9 to R22 is either a monovalent hydrocarbon group or a monovalent fluorinated hydrocarbon group,
R23 is either a divalent hydrocarbon group or a divalent fluorinated hydrocarbon group,
R9 and R10 are optionally bonded to each other,
R11 and R12 are optionally bonded to each other,
R13 and R14 are optionally bonded to each other,
R15 and R16 are optionally bonded to each other,
R17 and R18 are optionally bonded to each other,
R19 and R20 are optionally bonded to each other, and
any two or more of R21 to R23 are optionally bonded to each other.
